(19) United States
(12) Patent Application Publication
(10) Pub. No.: US 2022/0008388 A1
(43) Pub. Date: Jan. 13, 2022

(54) COMBINATION OF GABOXADOL AND LITHIUM FOR THE TREATMENT OF PSYCHIATRIC DISORDERS

(71) Applicant: Certego Therapeutics Inc., Farmingdale, NY (US)

(72) Inventors: Pavel Osten, New York City, NY (US); Kristin Kay Baldwin, New York City, NY (US); Robert DeVita, Westfield, NJ (US)

(21) Appl. No.: 17/295,870

(22) PCT Filed: Nov. 21, 2019
(86) PCT No.: PCT/US2019/062644

§ 371 Date: May 21, 2021

Related U.S. Application Data
(60) Provisional application No. 62/879,921, filed on Jul. 29, 2019.
(60) Provisional application No. 62/770,287, filed on Nov. 21, 2018.

(51) Int. Cl. A61K 31/407; A61K 33/00; A61P 25/18

(52) U.S. Cl. CPC A61K 31/407; A61P 25/18; A61K 33/00

(57) ABSTRACT

This disclosure reports on the discovery that low dose lithium can act in synergy with gaboxadol to enhance lithium's action on brain signaling activity. This combination of lithium and gaboxadol may greatly reduce the amount of lithium needed to treat many debilitating psychiatric disorders, such as bipolar disorder, depression, treatment resistant depression and suicidality, while reducing the often-serious side effects associated with high dose and chronic lithium treatment, especially nephrotoxicity, nephrogenic diabetes insipidus and chronic kidney disease. Co-administration of gaboxadol and lithium may also be useful for the treatment of refractory bipolar disorder, i.e. bipolar disorder which cannot be treated appropriately by administration of lithium alone. Gaboxadol may also prove useful as add-on therapy for the augmentation of the response to lithium in patients that do not respond to conventional lithium monotherapy.

### CROSS-REFERENCE TO RELATED APPLICATIONS

This application claims priority to Provisional Patent Application Nos. 62/770,287 filed on Nov. 21, 2018 and 62/879,921 filed on Jul. 29, 2019, the contents of which are incorporated by reference herein in their entireties.

### FIELD OF THE EMBODIMENTS

The present invention relates to compositions and methods of treating psychiatric disorders with a synergistic combination of lithium and gaboxadol.

### BACKGROUND OF THE EMBODIMENTS

Based on clinical practice guidelines lithium is, and has been since the 1960s, the first line treatment for mood stabilization and reduction of suicidality in bipolar disorders (BD), providing acute anti-manic therapeutic relief and preventing BD relapses to countless BD patients (Baldessarini et al., 2006; Cipriani et al., 2013; Kessing et al., 2018; Roberts et al., 2017; Sani et al., 2017; Severus et al., 2014). However, despite lithium's widespread use, it is not uncommon for patients to experience serious side effects from this medication.

For example, a first concern about lithium treatment is undoubtedly its very narrow therapeutic window, requiring the caregiver to maintain a serum concentration of typically 0.6-1.0 mmol/L for maintenance of bipolar disorder and higher levels of 1.0-1.2 mmol/L in acute mania treatment (Association, 2002; Gelenberg et al., 1989; Grandjean and Aubry, 2009). As lower levels are considered to be ineffective and lithium serum levels above this range can result in serious side-effects and toxicity, any treatment with lithium necessarily needs constant monitoring. This is especially true in BD patients who are pregnant because the associated increase in glomerular filtration rate can substantially reduce serum lithium levels to a point where there is a significant risk of BD relapse. Clinical strategy in pregnancy is therefore to increase lithium dose during pregnancy, thereby achieving higher serum levels during the early postpartum period which is often associated with an increased risk of relapse (Deligiannidis et al., 2014). However, close monitoring of lithium serum levels is essential because as the patient's kidney function returns to a lower glomerular filtration rate during the postpartum period, the associated increase in serum lithium levels can cause acute toxicity in the mother as well as in the infant (Horton et al., 2012; Wesseloo et al., 2017).

Acute lithium toxicity can present as non-convulsive status epilepticus, slowing of EEG alpha rhythm, pathological 3-10 Hz delta rhythm and diffuse spike discharges, life-threatening coma, hypothonia and hyporeflexia (Ivkovic and Stern, 2014; Madhusudhan, 2014; Megarbane et al., 2014; Schou et al., 1968).

Additionally, chronic side-effects associated with long-term maintenance lithium treatment include hypothyroidism, nephrogenic diabetes insipidus and significant nephrotoxicity and chronic kidney disease especially in patients already diagnosed with kidney failure (Davis et al., 2018a; Davis et al., 2018b). In an epidemiological study that examined reasons for discontinuing lithium therapy amongst BD patients, the majority (62%) of people stopped taking lithium because of the adverse events, primarily renal disease, diarrhea and/or tremor (Öhlund et al., 2018). In 2014 alone, there were 6,850 repotted cases of lithium toxicity in the United States. Lithium treatment therefore requires very careful monitoring and titration of serum lithium concentrations to achieve a long lasting therapeutic benefit.

Accordingly, there is an ongoing need for improved treatment options that mitigate the side effects associated with lithium treatment of many severe psychiatric disorders including bipolar disorder.

### SUMMARY OF THE EMBODIMENTS

This disclosure reports on the discovery that gaboxadol can act synergistically with lithium to enhance lithium's action on brain signaling activity. In particular, the combination of a sub-standard dose range of lithium (e.g. <600 mg per day) with gaboxadol reduces the amount of lithium needed to treat psychiatric disorders, such as bipolar disorder (both in acute mania and long-term maintenance), depression, treatment resistant depression and suicidality without the aforementioned side effects, especially chronic side-effects, such as nephrotoxicity and chronic kidney disease. Thus, co-administration of gaboxadol and a sub-standard dose of lithium lowers the risks of side-effects and facilitates the management of bipolar disorder and other psychiatric disorders responsive to lithium treatment. In addition, a standard dose range of lithium (e.g. 600 to 1800 mg, with a daily maximum dose of 2400 mg) also synergizes or, in certain embodiments, acts additively, with gaboxadol, indicating that adding gaboxadol to a standard dose of lithium may prove useful for the augmentation of the response to lithium in treatment-resistant patients who do not initially respond to conventional lithium monotherapy.

In a first aspect, a synergistic combination comprises gaboxadol and lithium, or a pharmaceutically acceptable salt of either or both compounds thereof.

In certain embodiments of the first aspect, the lithium is given in a sub-standard dose range that when administered daily to a subject in need thereof is ineffective at treating bipolar disorder, depression, treatment-resistant depression and suicidality.

In certain embodiments of the first aspect, the lithium is given in a sub-standard dose range that when administered daily to a subject in need thereof, is below the medically recommended dose for treating bipolar disorder, depression, treatment-resistant depression, or suicidality.

In certain embodiments of the first aspect, an animal equivalent of the sub-standard dose of lithium is ineffective at activating c-fos signalling in an animal model's brain as measured by Pharmacomapping.

In preclinical testing, the sub-standard lithium human dose range can be established and differentiated from the standard dose range, for example, by mapping lithium-induced brain activation, represented by the visualization of the induction of the immediate early gene (IEG) c-fos, in an animal such a mouse or rat, or by recording lithium-induced changes in an animal's electroencephalogram (EEG).

In certain embodiments of the first aspect, the sub-standard dose of lithium is in the range from about 50 to about 600 mg lithium carbonate per day.

In certain embodiments of the first aspect, gaboxadol is given at a low to medium human dose range that, when given to an animal such as a mouse or rat at an animal equivalent dose (AED), fails to evoke or evokes only a modest induction of c-fos activity in the brain as measured by Pharmacomapping.

In certain embodiments of the first aspect, the low dose of gaboxadol is in the range from about 5 to about 15 mg gaboxadol per day for an adult human and the medium dose of gaboxadol is in the range from about 15 to about 30 mg gaboxadol per day for an adult human.

In certain embodiments of the first aspect, the lithium is given at a standard dose range of lithium.

In certain embodiments of the first aspect, the standard dose range of lithium is in the range from about 600 to about 1800 mg, with a daily maximum dose of 2400 mg, of lithium carbonate per day for an adult human.

In certain embodiments of the first aspect, the gaboxadol is given as a high dose which, when given to an animal such as a mouse or rat at an animal equivalent dose (AED), evokes a strong induction of c-fos activity in the brain.

In certain embodiments of the first aspect, the high dose of gaboxadol is in the range from about 30 to about 300 mg gaboxadol per day for an adult human.

In certain embodiments of the first aspect, the amounts of lithium and gaboxadol administered daily to a subject in need thereof, are synergistically effective at inducing IEG c-fos signaling in at least one region of a subject's cortical brain selected from the group consisting of motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, and claustrum (CLA).

In certain embodiments of the first aspect, the amounts of lithium and gaboxadol administered daily to a subject in need thereof, are synergistically effective at inducing IEG c-fos signaling in at least two regions of a subject's cortical brain selected from the group consisting of motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, and claustrum (CLA).

In certain embodiments of the first aspect, the amounts of lithium and gaboxadol administered daily to a subject in need thereof, are synergistically effective at inducing IEG c-fos signaling in at least three regions of a subject's cortical brain selected from the group consisting of motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, and claustrum (CLA).

In certain embodiments of the first aspect, the amounts of lithium and gaboxadol administered daily to a subject in need thereof, are synergistically effective at inducing IEG c-fos signaling in at least one region of a subject's subcortical brain selected from the group consisting of hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS).

In certain embodiments of the first aspect, the amounts of lithium and gaboxadol administered daily to a subject in need thereof, are synergistically effective at inducing IEG c-fos signaling in at least two regions of a subject's subcortical brain selected from the group consisting of hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS).

In certain embodiments of the first aspect, the amounts of lithium and gaboxadol administered daily to a subject in need thereof, are synergistically effective at inducing IEG c-fos signaling in at least three regions of a subject's subcortical brain selected from the group consisting of hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS).

In certain embodiments of the first aspect, the amount of gaboxadol and lithium, when administered daily to a subject in need thereof, are synergistically effective at treating the subject's psychiatric disorder selected from the group consisting of bipolar disorder, depression, treatment resistant depression and suicidality.

In certain embodiments of the first aspect, the treatment of the subject's psychiatric disorder is effective at improving a score of at least one psychiatric rating scale specific for bipolar disorder, depression, treatment resistant depression or suicidality.

In certain embodiments of the first aspect, the gaboxadol and lithium, when administered to a subject diagnosed with depression, are synergistically effective at increasing the subject's Montgomery-Asberg Depression Rating Scale (MADRS) score.

In certain embodiments of the first aspect, the gaboxadol and lithium, when administered to a subject in need thereof, are synergistically effective at increasing a score of at least one psychiatric rating scale specific for bipolar disorder, depression, treatment resistant depression or suicidality.

In certain embodiments of the first aspect, the gaboxadol and lithium, when administered to a subject in need thereof, are synergistically effective at increasing a score of at least two psychiatric rating scales specific for bipolar disorder, depression, treatment resistant depression or suicidality.

In certain embodiments of the first aspect, the gaboxadol and lithium, when administered to a subject in need thereof, are synergistically effective at increasing a score of at least three psychiatric rating scales specific for bipolar disorder, depression, treatment resistant depression or suicidality.

In certain embodiments of the first aspect, the lithium, when administered daily to a subject in need thereof, is in an amount sufficient to maintain the serum level of lithium in the range of about 0.2 to about 1.2 mmol/L.

In certain embodiments of the first aspect, the lithium, when administered daily to a subject in need thereof, is in an amount sufficient to maintain the subject's serum level of lithium in the range of about 0.4 to about 0.8 mmol/L.

In a second aspect, a pharmaceutical composition is disclosed that comprises any one of the preceding embodiments of the synergistic combination of lithium and gaboxadol.

In certain embodiments of the second aspect, the pharmaceutical composition is in the form of a single tablet for oral consumption.

In certain embodiments of the second aspect, the pharmaceutical composition is in a form of a controlled release formulation.

In certain embodiments of the second aspect, the pharmaceutical composition further comprises one or more inert pharmaceutically acceptable excipients.

In certain embodiments of the second aspect, the pharmaceutical composition is in the form of a single dosage unit having separate compartments for the lithium and gaboxadol or a pharmaceutically acceptable salt of either or both compounds thereof.

In a third aspect, a kit is disclosed that comprises any one of the preceding pharmaceutical compositions.

In a fourth aspect, a method is disclosed for treating a subject in need thereof comprising administering any one of the previous embodiments of the synergistic combination of lithium and gaboxadol.

In certain embodiments of the fourth aspect, the subject is diagnosed with a psychiatric disorder.

In certain embodiments of the fourth aspect, the psychiatric disorder is chosen from bipolar disorder, depression, treatment-resistant depression or suicidality.

In certain embodiments of the fourth aspect, the combination reduces at least one adverse side effect selected from the group consisting of nephrotoxicity, nephrogenic diabetes insipidus, chronic kidney disease, diarrhea, hand tremor, increased thirst, increased urination, vomiting, weight gain, impaired memory, poor concentration, drowsiness, muscle weakness, hair loss, acne and decreased thyroid function.

In certain embodiments of the fourth aspect, the combination reduces at least two adverse side effects selected from the group consisting of nephrotoxicity, nephrogenic diabetes insipidus, chronic kidney disease, diarrhea, hand tremor, increased thirst, increased urination, vomiting, weight gain, impaired memory, poor concentration, drowsiness, muscle weakness, hair loss, acne and decreased thyroid function.

In certain embodiments of the fourth aspect, the combination reduces at least three adverse side effects selected from the group consisting of nephrotoxicity, nephrogenic diabetes insipidus, chronic kidney disease, diarrhea, hand tremor, increased thirst, increased urination, vomiting, weight gain, impaired memory, poor concentration, drowsiness, muscle weakness, hair loss, acne and decreased thyroid function.

In a fifth aspect, a method for treating a human diagnosed with bipolar disorder, depression, treatment-resistant depression or acute suicidality is disclosed that comprises administering a synergistic combination of gaboxadol at a dose ranging from about 5 to about 300 mg/day, contemporaneously with lithium at a dose from about 50 mg to about 1800 mg lithium carbonate, with a maximum daily dose of 2400 mg [for a 60 kg human]; or from about 0.8 mg/kg to about 30 mg/kg, with a maximum dose of 40 mg/kg, of lithium carbonate; or in an amount sufficient to achieve a lithium serum concentration of about 0.2 to 1.2 mmol/L; wherein the combination is administered at least once per day.

In a sixth aspect, a method for treating a human diagnosed with an acute form of bipolar disorder, depression, treatment-resistant depression or suicidality comprising administering a synergistic combination of gaboxadol at a dose in the range of from about 5 mg to about 150 mg/day, contemporaneously with lithium at a dose of from about 300 mg to about 1800 mg lithium carbonate/day [for a 60 kg human]; or in an amount sufficient to achieve a lithium serum concentration of 0.4 to 1.2 mmol/L, wherein the combination is administered at least once per day.

In a seventh aspect, a method for treating a patient diagnosed with an acute form of bipolar disorder, depression, treatment-resistant depression or suicidality comprising administering a synergistic combination of gaboxadol at a dose in the range of from about 5 mg to about 150 mg/day, contemporaneously with lithium at a dose of from about 50 mg to about 900 mg lithium carbonate [for a 60 kg human]; or in an amount sufficient to achieve a lithium serum concentration of about 0.2 to 1.0 mmol/L; wherein the combination dose is administered at least once per day.

In an eighth aspect, a method for treating a patient diagnosed with an acute form of bipolar disorder, depression, treatment-resistant depression or suicidality comprising administering an additive combination of gaboxadol at a dose in the range of from about 5 mg to about 150 mg/day, contemporaneously with lithium at a dose from about 50 mg to about 900 mg lithium carbonate [for a 60 kg human]; or in an amount sufficient to achieve a lithium serum concentration of about 0.2 to 1.0 mmol/L, wherein the combination dose is administered at least once per day.

In a ninth aspect, a use of gaboxadol and lithium in the preparation of a fixed dose combination medicament is disclosed, wherein the lithium is present in a range from about 10 mg to about 300 mg [lithium carbonate 50 mg to about 1800 mg]; and wherein the gaboxadol is present in a range from about 5 mg to about 150 mg for treatment of a human patient diagnosed with bipolar disorder, depression or acute suicidality.

In a tenth aspect, a use of a fixed dose combination comprising lithium and gaboxadol in unit dosage form is disclosed wherein the lithium is present in a range from about 10 mg to about 360 mg [lithium carbonate 50 mg to about 1800 mg] and wherein the gaboxadol is present in a range from about 5 mg to about 150 mg for the treatment of a human patient diagnosed with bipolar disorder, depression or acute suicidality.

In a eleventh aspect, a use of a fixed dose combination comprising lithium and gaboxadol in unit dosage form for once daily administration is disclosed, wherein the gaboxadol is present from about 5 mg to about 150 mg, and the lithium for the first week is present in the range of from about 40 mg to about 360 mg [lithium carbonate 200 mg to about 1800 mg], and after the first week the lithium is present in the range of from about 10 mg to about 180 mg [lithium carbonate 50 mg to about 900 mg], for the treatment of a human patient diagnosed with bipolar disorder, depression or acute suicidality.

In a twelfth aspect, a use of a synergistic combination of gaboxadol and lithium, or a pharmaceutically acceptable salt of either or both compounds thereof, is disclosed for reducing one or symptoms of bipolar disorder, depression, or suicidality.

In a thirteenth aspect, a use of a synergistic combination of gaboxadol and lithium, or a pharmaceutically acceptable salt of either or both compounds thereof, is disclosed in the manufacture of a medicament for reducing one or symptoms of bipolar disorder, depression, or suicidality.

### BRIEF DESCRIPTION OF THE DRAWINGS

FIG. 1 shows exemplary whole-brain pharmacomaps representing drug-evoked brain activation in the mouse.

(A) Mice were treated with a drug or vehicle solution for the control group using either intraperitoneal (i.p.), per oral (p.o.). subcutaneous (s.c.), intramuscular (i.m.) or intravenous (i.v.) delivery.

(B) Treatment with the drug led to the induction of the immediate early gene c-fos expression in activated neurons that peaked within about 1.5 to about 3 hours depending on the drug's pharmacokinetics.

(C) After the aforementioned c-fos induction period, the mice were sacrificed and the c-fos induction was visualized using whole-brain immunostaining. The brains were then chemically cleared and imaged by light-sheet fluorescent microscopy (LSFM).

(D) The whole-brain scans are depicted as serial section datasets with an XYZ resolution of about 4×4×5 microns.

(E) The c-fos-positive cells were detected in these datasets using custom algorithms.

(F) The whole-brain distribution of the detected c-fos-positive cells was then represented in 3D as a spatial map of centroid points in the 3D space of the mouse brain.

(G) This 3D map distribution was registered to a reference mouse brain and spatially voxelized using overlapping 150-micron sphere voxels.

(H) Finally, the drug-evoked Pharmacomap® was generated by a statistical comparison of the c-fos-positive cell distributions of the drug-treated and control vehicle-treated mice, typically using 6 animals per group.

FIG. 2 shows exemplary lithium dose-curve pharmacomaps.

White color indicates the spatial areas having significant lithium-evoked induction of c-fos activity in a mouse brain. The broad activation pattern induced by lithium with increasing dose was localized, for example, to the following anatomical structures: Cortex: prelimbic (PL) and infralimbic (ILA) cortex, piriform cortex (PIR), associational visceral (VISC), gustatory (GU), agranular insular (AIp) cortical areas, retrosplenial (RSP), motor (MO), somatosensory (SS), auditory (AUD), visual (VIS), temporal associational (Tea), perirhinal (PERI) and entorhinal (ENT), and ectorhinal (ECT) cortex; Basal ganglia: the nucleus accumbens (ACB), the anterior part of the bed nuclei of the stria terminalis (BSTa), cortical amygdala and central amygdala (CEA); Midline thalamus: paraventricular nucleus (PVT), intermediodorsal nucleus (IMB), central medial nucleus (CM), and rhomboid nucleus (RH); Midbrain: geniculate complex (MG); Brainstem: locus coeruleus (LC).

FIG. 3 shows exemplary lithium induced brain activation is similar to that of gaboxadol.

White color indicates the spatial areas of significant lithium-evoked induction of c-fos activity in a mouse brain. The broad activation pattern evoked by lithium at a dose of 300 mg/kg (top row; human equivalent dose about 1500 mg) appears similar to the effect of gaboxadol at 20 mg/kg (bottom row; human equivalent dose about 100 mg), including the following anatomical structures: Cortex: infralimbic (ILA) cortex, piriform cortex (PIR), associational visceral (VISC), gustatory (GU), agranular insular (AIp) cortical areas, retrosplenial (RSP), motor (MO), somatosensory (SS), auditory (AUD), visual (VIS), temporal associational (Tea), perirhinal (PERI) and entorhinal (ENT), and ectorhinal (ECT) cortex; Basal ganglia: the nucleus accumbens (ACB), the anterior part of the bed nuclei of the stria terminalis (BSTa), cortical amygdala and central amygdala (CEA); Midline thalamus: paraventricular nucleus (PVT), intermediodorsal nucleus (IMB), central medial nucleus (CM), and rhomboid nucleus (RH); Midbrain: geniculate complex (MG); Brainstem: locus coeruleus (LC).

FIG. 4 shows an exemplary synergistic effect from the co-administration of low dose gaboxadol and sub-standard dose of lithium

White color indicates the spatial areas of significant lithium-evoked induction of c-fos activity in a mouse brain. While neither lithium at 85 mg/kg (top row; human equivalent dose about 425 mg), nor gaboxadol at 3 mg/kg (middle row; human equivalent dose about 15 mg) induced any brain activation on their own, the combination of these low doses induced a prominent and broad activation (bottom row) indicating a synergy between the two compounds within multiple anatomical brain structures, including: Cortex: infralimbic (ILA) cortex, piriform cortex (PIR), associational visceral (VISC), gustatory (GU), agranular insular (AIp) cortical areas, retrosplenial (RSP), motor (MO), somatosensory (SS), auditory (AUD), visual (VIS), temporal associational (Tea), perirhinal (PERI) and entorhinal (ENT), and ectorhinal (ECT) cortex; Basal ganglia: the nucleus accumbens (ACB), the anterior part of the bed nuclei of the stria terminalis (BSTa), cortical amygdala and central amygdala (CEA); Midline thalamus: paraventricular nucleus (PVT), intermediodorsal nucleus (IMB), central medial nucleus (CM), and rhomboid nucleus (RH); Midbrain: geniculate complex (MG); Brainstem: locus coeruleus (LC). The weak pattern of inhibition (green color) seen across the caudoputamen (CP) and hippocampus (HIPP) suggest a modest sedation induced by the two compounds.

FIG. 5A shows an exemplary synergistic and additive brain activation effect of co-administration of medium dose gaboxadol and standard dose of lithium.

White color indicates the spatial areas of significant lithium-evoked induction of c-fos activity in a mouse brain. While lithium at 150 mg/kg (top row; human equivalent dose about 750 mg) and gaboxadol at 6 mg/kg (middle row; human equivalent dose about 30 mg) evoked moderate brain activation on their own, including infralimbic (ILA) cortex, the anterior part of the bed nuclei of the stria terminalis (BSTa), locus coerules (LC) and some additional cortical areas, the combination of these two doses evoked a considerably more prominent activation (bottom row) further demonstrating a synergy and additive action between the two compounds, including the following anatomical structures: Cortex: infralimbic (ILA) cortex, piriform cortex (PIR), associational visceral (VISC), gustatory (GU), agranular insular (AIp) cortical areas, retrosplenial (RSP), motor (MO), somatosensory (SS), auditory (AUD), visual (VIS), temporal associational (Tea), perirhinal (PERI) and entorhinal (ENT), and ectorhinal (ECT) cortex; Basal ganglia: the nucleus accumbens (ACB), the anterior part of the bed nuclei of the stria terminalis (BSTa), cortical amygdala and central amygdala (CEA); Midline thalamus: paraventricular nucleus (PVT), intermediodorsal nucleus (IMB), central medial nucleus (CM), and rhomboid nucleus (RH); Midbrain: geniculate complex (MG); Brainstem: locus coeruleus (LC). The weak pattern of inhibition (green color) seen across the caudoputamen (CP) and hippocampus (HIPP) suggest a modest sedation induced by the two compounds.

FIG. 5B shows an exemplary synergistic and additive brain activation effect of co-administration of medium dose of gaboxadol and standard dose of lithium.

White color indicates the spatial areas of significant lithium-evoked activation in a mouse brain. While lithium at 200 mg/kg (top row; human equivalent dose about 1000 mg) and gaboxadol at 6 mg/kg (middle row; human equivalent dose about 30 mg) evoked moderate brain activation on their own, including infralimbic (ILA) cortex, the anterior part of the bed nuclei of the stria terminalis (BSTa), locus coerules (LC) and some additional cortical areas, the combination of these two doses evoked a considerably more prominent activation (bottom row) further demonstrating a synergy between the two compounds, including the following anatomical structures: Cortex: infralimbic (ILA) cortex, piriform cortex (PIR), associational visceral (VISC), gustatory (GU), agranular insular (AIp) cortical areas, retrosplenial (RSP), motor (MO), somatosensory (SS), auditory (AUD), visual (VIS), temporal associational (Tea), perirhinal (PERI) and entorhinal (ENT), and ectorhinal (ECT) cortex; Basal ganglia: the nucleus accumbens (ACB), the anterior part of the bed nuclei of the stria terminalis (BSTa), cortical amygdala and central amygdala (CEA); Midline thalamus: paraventricular nucleus (PVT), intermediodorsal nucleus (IMB), central medial nucleus (CM), and rhomboid nucleus (RH); Midbrain: geniculate complex (MG); Brainstem: locus coeruleus (LC). The weak pattern of inhibition (green color) seen across the caudoputamen (CP) and hippocampus (HIPP) suggest a modest sedation induced by the two compounds.

FIG. 6 shows an exemplary synergistic behavioral effect of co-administration of a low dose of gaboxadol and sub-standard dose of lithium.

Mice were pretreated with vehicle (saline) or a sub-therapeutic dose of lithium (lithium 14.1 mg/kg; human equivalent dose about 70 mg) or low dose of gaboxadol (gaboxadol−3 mg/kg; human equivalent dose about 15 mg) or a combination of lithium and gaboxadol (lithium 14.1 mg/kg+gaboxadol−3 mg/kg) for 20 min before treatment with d-amphetamine at 3.5 mg/kg. While the locomotion, expressed as ambulatory counts (i.e. the number of beam breaks while ambulatory) was comparable between the four groups for the initial 20 min, the treatment with d-amphetamine induced a sharp increase in locomotion in the vehicle treated animals (top dark blue line), that was not moderated by lithium at 14.1 mg/kg alone (second from top orange line) and only modestly (Anova p value=0.007, Fisher's PLSD test p=0.6) by gaboxadol at 3 mg/kg alone (second from bottom yellow line), whereas a combination of lithium at 14.1 mg/kg and gaboxadol at 3 mg/kg (bottom light blue line) showed a pronounced moderation in locomotion (Anova p value=0.007, Fisher's PLSD test p=<0.01), demonstrating synergy between the two molecules.

### DESCRIPTION OF THE PREFERRED EMBODIMENTS

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### 1) Definitions

Unless defined otherwise, all technical and scientific terms used herein have the same meanings as commonly understood by one of skill in the art to which the disclosure herein belongs.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In certain embodiments, the term “about” or “approximately” as used herein means within an acceptable error range for the particular value as determined by one of ordinary skill in the art, which will depend in part on how the value is measured or determined, i.e., the limitations of the measurement system. In certain embodiments, “about” can mean within 3 or more than 3 standard deviations, per the practice in the art. In certain embodiments, particularly with respect to biological systems or processes, the term can mean within an order of magnitude, preferably within 5-fold, and more preferably within 2-fold, of a value.

In certain embodiments, when the term “about” or “approximately” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%. In certain embodiments, the term “about” is used to modify a numerical value above and below the stated value by a variance of 0.1%.

When a range of values is listed herein, it is intended to encompass each value and sub-range within that range. For example, “1-5 ng” or “from about 1 ng to about 5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, “contemporaneously” refers to the length of time between the administration of gaboxadol and lithium taken separately. In certain embodiments, the “coadministration” of gaboxadol and lithium refers to the contemporaneous administration of gaboxadol and lithium. In certain embodiments, an administration of gaboxadol and lithium is contemporaneous if gaboxadol is administered to a patient in need thereof within about 5 minutes, about 30 minutes, about 1 hour, about 2 hours, about 3 hours, about 4 hours, about 5 hours, about 6 hours, about 7 hours, about 8 hours, about 9 hours, about 10 hours, about 11 hours or about 12 hours of the administration of lithium. In certain embodiments, gaboxadol is administered to a patient in need thereof within about 2 hours of the administration of lithium. In certain embodiments, an administration of gaboxadol and lithium is contemporaneous if lithium is administered to a patient in need thereof within about 5 minutes, about 30 minutes, about 1 hour, about 2 hours, about 3 hours, about 4 hours, about 5 hours, about 6 hours, about 7 hours, about 8 hours, about 9 hours, about 10 hours, about 11 hours or about 12 hours of gaboxadol. In certain embodiments, lithium is administered to a patient in need thereof within about 2 hours of the administration of gaboxadol. In certain embodiments, a contemporaneous administration of lithium and gaboxadol can include lithium being administered simultaneously with gaboxadol either as separate doses or in a combined dose.

As referred to herein, unless stated otherwise, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

As used herein, unless stated otherwise, the term subject refers to a mammal, e.g., a human, mouse, rat, guinea pig, dog, cat, horse, cow, pig, or non-human primate, such as a monkey, chimpanzee, or baboon. The terms “subject” and “patient” are used interchangeably. In certain embodiments, a subject is a human suffering from a psychiatric disorder such as depression or bipolar disorder. In certain embodiments, the subject is a human. In certain embodiments, the human is a pediatric human. In certain embodiments, the subject is an adult human.

The terms “effective amount” or “therapeutically effective amount” as used herein, unless stated otherwise, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In some embodiments, the effective amount is a dose that is generally effective in alleviating, reducing, noticeably reducing, or eliminating, symptoms associated with bipolar disorder or mania. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

As used herein, unless stated otherwise, the term “treatment-resistant” is used as that term is understood by one skilled in the art, and as used in the present invention, means a lack of therapeutic response after at least one trial of an antidepressant at an adequate dose for about six weeks.

The terms “administer,” “administering,” “administration,” and the like, as used herein, refer to the methods that are used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In some embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

A “cocrystal” as used herein refers to a multiple component crystal containing two or more non-identical compounds (cocrystal precursors) in a stoichiometric ratio (1:1) or a ratio of (2:1), each of which is solid under ambient conditions (i.e., 22° C., 1 atmosphere of pressure) when in their pure form.

As used herein, the terms “treat,” “treatment” or “treating” refer to therapeutic treatments, wherein the object is to reverse, alleviate, ameliorate, inhibit, slow down or stop the progression or severity of a psychiatric disorder, e.g., depression, treatment resistant depression acute suicidality and bipolar disorder. The term “treating” includes reducing or alleviating at least one adverse effect or symptom of the psychiatric disorder. Treatment is generally “effective” if one or more symptoms or clinical markers are decreased. Alternatively, treatment is “effective” if the progression of the disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” also includes providing relief from the symptoms or side-effects of a disorder, e.g. a psychiatric disorder (including palliative treatment).

As used herein, unless otherwise specified, the terms “prevent,” “preventing” and “prevention” contemplate an action that occurs before a patient begins to suffer from the specified disease or disorder, which inhibits or reduces the severity or symptoms of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder or changing the way that a patient responds to the disease or disorder.

As used herein, “c-fos signaling,” “c-fos activity,” “c-fos expression,” “c-fos activation,” “c-fos gene expression,” “c-fos signaling activity,” “brain signaling activity” or “c-fos immediate early gene (IEG) expression” are used interchangeably and refer to the activation of the expression of an immediate early gene (IEG), e.g. c-fos, as measured, for example, by immunohistochemistry, in situ hybridization of c-fos-specificationific probes, GFP expression in c-fos-GFP mice or by pharmacomapping as disclosed herein (see Examples 1-5).

As used herein, unless stated otherwise, the term “synergy” or “synergism” or “synergistically” or “synergistic” refers to the interaction of lithium and gaboxadol so that their combined effect is greater than the sum of their individual effects. In certain embodiments, a synergistic combination of lithium and gaboxadol is effective at treating, preventing and/or managing a psychiatric disorder, including, but not limited to bipolar disorder, depression, treatment-resistant depression and suicidality.

As used herein, unless stated otherwise, the term “additive” or “additively” or “additive effect” or “additive action” refers to the interaction of lithium and gaboxadol so that their combined effect is equal to the sum of their individual effects. In certain embodiments, an additive combination of lithium and gaboxadol is effective at treating, preventing and/or managing a psychiatric disorder, including, but not limited to bipolar disorder, depression, treatment-resistant depression and suicidality.

As used herein, and unless otherwise indicated, the term “low induction of c-fos activity in the brain” refers to gaboxadol-evoked or lithium-evoked or gaboxadol+lithium combination-evoked induction of c-fos that includes one to two cortical areas, such as anterior cingulate (ACA) and retrosplenial (RSP) cortex and/or one to three subcortical areas, such as bed nuclei stria terminalis (BST), central amygdala (CEA), and locus coeruleus (LC).

As used herein, and unless otherwise indicated, the term “moderate induction of c-fos activity in the brain” refers to gaboxadol-evoked or lithium-evoked or gaboxadol+lithium combination-evoked induction of c-fos that includes three to six cortical areas, such as ACA, RSP, gustatory (GU), visceral (VISC), auditory (AUD) and visual (VIS) cortex and/or four to six subcortical areas, such as BST, CEA, LC, nucleus of reunions (RE), romboid nucleus (RH) and central medial nucleus (CM) of the thalamus.

As used herein, and unless otherwise indicated, the term “strong induction of c-fos activity in the brain” refers to gaboxadol-evoked or lithium-evoked or gaboxadol+lithium combination-evoked induction of c-fos that includes more than 6 cortical areas, such as ACA, RSP, GU, VISC, AUD, VIS, motor (MO), agranular insular (AI), somatosensory (SS), prelimbic (PL) and infralimbic (ILA), parietal (PTL), temporal associational (TEa), ectorhinal (ECT), entorhinal (ENT), perirhinal (PERI), and piriform (PIR) cortex and claustrum (CLA) and/or more than 6 subcortical areas, such as BST, CEA, LC, RE, RH, CM, hippocampal CA1 region, cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, and nucleus of the solitary tract (NTS).

As used herein, and unless otherwise indicated, the term “sub-standard dose” for lithium refers to human doses in the range of about 50 to about 600 mg for an adult human) that would be expected to lack therapeutic efficacy in lithium monotherapy of bipolar disorder, depression, treatment-resistant depression and suicidality.

As used herein, and unless otherwise indicated, the term “standard dose” for lithium refers to human doses in the range of 600 to 1800 mg, with a maximum daily dose of 2400 mg mg (for an adult human), lithium carbonate that would be expected to be therapeutically effective as a lithium monotherapy of bipolar disorder, depression, treatment-resistant depression and suicidality.

As used herein, and unless otherwise indicated, the term “low dose” for gaboxadol refers to a human dose in the range of 5 to about 15 mg for an adult human or an animal equivalent dose of about 1 to about 3 mg/kg that, when administered to an animal model such as a mouse or rat, does not induce an activation of immediate early c-fos gene (IEG) expression in a mouse brain.

As used herein, and unless otherwise indicated, the term “medium dose” for gaboxadol refers to a human dose in the range of 15 to about 30 mg for an adult human or an animal equivalent dose of about 3 to about 6 mg/kg that, when administered to an animal model such as a mouse or rat. induces moderate activation of c-fos immediate early gene (IEG) expression in a mouse brain.

As used herein, and unless otherwise indicated, the term “high dose” for gaboxadol refers to a human dose in the range of about 30 to about 100 mg for an adult human or an animal equivalent dose of about 6 to about 20 mg/kg that, when administered to an animal model such as a mouse or rat, induces a strong activation of c-fos immediate early gene (IEG) expression in a mouse brain. As used herein, a dose expressed as mg/kg refers to number of milligrams of medication per kilogram of the body weight of the subject taking the medication.

### 2) Lithium Monotherapy

Lithium was first described as a mood stabilizer by the Australian psychiatrist John Cade in 1949 for the treatment of acute mania (Cade J F Med J Aust. 1949; 2(10):349-52). Approved in 1970 by the U.S. Food and Drug Administration, the mechanism of action of lithium remains a mystery, though it is has been proposed that lithium's action stems, at least in part, from the ability of the Li+ ion to inhibit glycogen synthase kinase 3 and inositol monophosphatase by displacing magnesium, a cofactor essential for enzymatic activity (see, for example, U.S. Pat. No. 9,265,764, the content of which is incorporated by reference herein in its entirety). Lithium is now widely prescribed for the treatment of bipolar disorder, unipolar depression, treatment resistant depression and suicide prevention.

a) Treatment of Bipolar Disorder

Bipolar disorder is a mood disorder characterized by unusually intense emotional states that occur in distinct periods called “mood episodes.” An overly elated or overexcited state is called a manic episode, and an extremely sad or hopeless state is called a depressive episode. Individuals suffering from bipolar disorder experience manic episodes, also commonly experience depressive episodes or symptoms, or mixed episodes in which features of both mania and depression are present at the same time. These episodes are usually separated by periods of “normal” mood, but in some individuals, depression and mania may rapidly alternate, known as rapid cycling. Extreme manic episodes can sometimes lead to psychotic symptoms such as delusions and hallucinations. Patients affected by bipolar disorder have had at least one manic or hypomanic (mild mania) episode. Patients with full manias and depression are indicated as having “bipolar I disorder”. Patients with hypomania and depression are described as having “bipolar II disorder.” Onset of episodes tends to be acute, with symptoms developing over days to weeks.

Symptoms of mania or a manic episode include both mood changes and behavioral changes. Mood changes include the following: a long period of feeling “high,” or an overly happy or outgoing mood; and extremely irritable mood, agitation, feeling “jumpy” or “wired.” Behavioral changes include the following: talking very fast, jumping from one idea to another, having racing thoughts; being easily distracted; increasing goal-directed activities, such as taking on new projects; being restless; sleeping little; having an unrealistic belief in one's abilities; behaving impulsively and taking part in a lot of pleasurable; and high-risk behaviors, such as spending sprees, impulsive sex, and impulsive business investments.

Symptoms of depression or a depressive episode include both mood changes and behavioral changes. Mood changes include the following: a long period of feeling worried or empty; and loss of interest in activities once enjoyed, including sex. Behavioral Changes include the following: feeling tired or “slowed down”; having problems concentrating, remembering, and making decisions; being restless or irritable; changing eating, sleeping, or other habits; and thinking of death or suicide, or attempting suicide.

In adult BP patients, the dose of lithium needed to achieve treatment efficacy in acute mania typically begins with a dose between 600-900 mg lithium carbonate per day or approximately 10 to 15 mg/kg for an adult human, which is gradually increased up to 1800 mg or approximately 30 mg/kg for an adult human of lithium carbonate per day. Lithium has been traditionally given in 2-4 divided doses, but a single evening dose has also been used and was shown to have a comparable therapeutic efficacy with improved compliance and even lesser renal adverse effects (Carter et al., 2013; Ljubicic et al., 2008; Singh et al., 2011). The maximum daily dose usually should not exceed 2400 mg lithium carbonate or approximately 40 mg/kg for an adult human. The typical therapeutic concentration of lithium in the serum for acute treatment of manic episodes is in the range of 0.6 to 1.2 mmol/L and the number of patients with a positive therapeutic response increases as the serum lithium concentration increases.

For long-term control of BD in adults, the recommended dose is between 900 to 1500 mg lithium carbonate/day or approximately 15 to 25 mg/kg for an adult human adult orally per day. Lithium concentrations in the blood considered safe for BD maintenance treatment are in the range as low as 0.4 mmol/L to 1.2 mmol/L, with the higher end of the range improving the likelihood of effective prophylactic therapy. However, given the concerns about side-effects caused by the long term use of lithium, the lower target ranges of 0.4 to 0.8 mmol/L are often used. In contrast, concentrations between 1.2-2.5 mmol/L may be associated with mild toxicity, concentrations between 2.5 and 3.5 mmol/L result in severe toxicity, and concentrations greater than 3.5 mmol/L can be life-threatening.

In pediatric BD patients, dosing of lithium is within the range estimates for adults when accounting for body size, i.e. 20-30 mg per kg of lithium carbonate per day in an acute treatment and 10 to 25 mg of lithium carbonate per kg for chronic treatment.

Dosing in older patients must be carefully monitored to account for age-related lower renal function, leading to two to threefold decreases in doses needed to achieve the desired serum concentration (Rej et al., 2014).

Lithium treatment and monitoring during pregnancy is particularly challenging because increased glomerular filtration rate leads to a substantial reduction of lithium levels and risk of BD relapse. Clinical strategy in pregnancy is therefore to increase lithium dose during pregnancy and in addition achieve higher serum levels during the early postpartum period that is associated with a strongly increased risk of relapse (Deligiannidis et al., 2014). However, as the kidney functions return to normal in the postpartum period, high lithium doses can lead to acute toxicity to the mother as well as to the infant (Horton et al., 2012; Wesseloo et al., 2017).

In adults and children 12 years of age or older suffering from acute mania, the oral dose of extended-release tablets can be 900 mg 2 times a day, or 600 mg 3 times a day. For the long-term treatment of mania, in adults and children 12 years of age or older the oral dose can be 600 mg 2 times a day, or 3 times a day up to 1200 mg per day. Treatment of mania in children younger than 12 years of age is not recommended.

b) Treatment of Unipolar Depression and Suicide Prevention

Unipolar depression or major depressive disorder (MDD) is used as that term is understood in art, and refers to a diagnosis that is guided by diagnostic criteria listed in DSM-IV or ICD-10, or in similar nomenclatures (DSM IV-TR—Desk reference to the diagnostic criteria from DSM-IV-TR, American Psychiatric Association, Washington D.C. 2000; Kaplan, H. I. et al. Kaplan and Sadock's Synopsis of Psychiatry (8th edition) 1998 Williams & Wilkins, Baltimore). Unipolar depression is a major clinical problem with lifetime prevalence in Western cultures estimated to be between 4%-12%. Although approximately 70% of patients respond to treatment with antidepressants, up to 75% have a recurrence within 10 years and a very high proportion of sufferers remain undiagnosed and untreated. The unipolar connotes a difference between major depression and bipolar depression, which refers to an oscillating state between depression and mania. Instead, unipolar depression is solely focused on the “lows” characterized by a rumination on negative emotions. DSM IV requires for the diagnosis of major depression the presence of a major depressive episode. This in turn consists of at least five of the nine symptoms present during the same 2-week period, of which depressed mood or loss of interest or pleasure has to be one of the symptoms. Changes in weight/appetite, sleep, energy, psychomotor retardation or agitation, guilt, decreased concentration, suicidality are the other symptoms. It should also be noted that depression is not the only psychiatric disorder leading to suicide. Other disorders like bipolar disorder, psychotic disorders (like schizophrenia), anxiety disorders (including panic disorders, OCD, PTSD), alcohol and drug addictions, and personality disorders may also lead to suicide.

Lithium is also used as an adjunct treatment and for reducing suicidality in unipolar depression, especially in a population of patients with treatment-resistant depression (Cipriani et al., 2013; Cipriani et al., 2005; Roberts et al., 2017). In addition, lithium has also been recommended in the prophylaxis of recurrent unipolar depressive episodes in this population, with the suggestion to start the prophylactic life-long treatment after the occurrence of 2 episodes of severe depression with suicidal risk within 5 years (Abou-Saleh et al., 2017; Baldessarini et al., 2003; Post, 2018; Tiihonen et al., 2016; Toffol et al., 2015). Remarkably, lithium appears to have an anti-suicide effect even at very low concentrations in drinking water, typically less than 150 μg/L (Ando et al., 2017; Vita et al., 2015).

Despite its well-established efficacy in the treatment of BD, lithium treatment has several disadvantages. The therapeutically effective window is very narrow which means even modest changes in serum concentration can have significant toxicity leading to, for example, nephrotoxicity accompanied with extreme thirst, nausea, vomiting, diarrhea, drowsiness, muscle weakness, tremor, lack of coordination, hallucinations, seizures (blackout or convulsions), vision problems, dizziness, fainting, slow heart rate, or fast or uneven heartbeats. In addition, long-term therapeutic maintenance amongst BD patients can be quite variable, with only approximately 30% of patients showing good long-term efficacy (Scott et al., 2017). Therefore, the development of a lithium form with good therapeutic efficacy in a broader population of BD would dramatically improve BD treatment options.

3) Gaboxadol Monotherapy

Gaboxadol, gaboxadolum or THIP (4, 5, 6, 7-tetrahydroisoxazolo (5, 4-c) pyridin-3-ol; C₆H₈N₂O₂; Cas Number: 64603-91-4; PubChem CID: 3448) is a selective GABAA receptor agonist with a preference for δ-subunit containing GABAA receptors having the structure of:

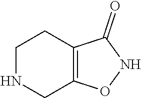

“Gaboxadol” is intended to include any form of the compound, such as the base (zwitter ion), pharmaceutically acceptable salts, e.g. pharmaceutically acceptable acid addition salts, hydrates or solvates of the base or salt, as well as anhydrates, and also amorphous, or crystalline forms.

Typically, the medicament can be in a solid oral dose form, such as tablets or capsules, or a liquid oral dose form. Thus, a typical embodiment is use of gaboxadol for preparing a medicament in an oral dose form comprising an effective amount of the gaboxadol from 2.5 mg to 100 mg. Preferably, the gaboxadol is in a crystalline form. Further embodiments of the medicament comprises an effective amount of gaboxadol from about 2.5 mg to about 100 mg, such as 2.5 mg to 4 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg, e.g. 2.5 mg, 5 mg, 7.5 mg, 10 mg, 12.5 mg, 15 mg, 20 mg, 40 mg or 50 mg. A typical embodiment being about 15 mg to about 50 mg of crystalline gaboxadol, such as the hydrochloride of gaboxadol.

Methods for the preparation of solid pharmaceutical preparations are well known in the art. Tablets may thus be prepared by mixing the active ingredients with ordinary adjuvants and/or diluents and subsequently compressing the mixture in a convenient tableting machine. Examples of adjuvants or diluents comprise: corn starch, lactose, talcum, magnesium stearate, gelatin, lactose, gums, and the like. Any other adjuvant or additive such as colorings, aroma, preservatives, etc. may also be used provided that they are compatible with the active ingredients.

Examples of gaboxadol formulations, or pharmaceutically acceptable salts thereof, are disclosed in the following patent publications: WO2018144827, US20110082171, US20090048288, WO2006118897, WO2006102093, US20050137222, WO2002094225, WO2001022941, the contents of which are incorporated by reference herein in their entireties.

In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia. The development was discontinued after the compound failed to show significant effects in sleep onset and sleep maintenance in a three-month efficacy study.

A clinical trial to investigate the efficacy of gaboxadol in the treatment of symptoms of Angelman Syndrome (a developmental disorder) sponsored by Ovid Therapeutics Inc. (ClinicalTrials.gov Identifier: NCT02996305) is currently underway. Patent applications on related subject matter include U.S. Pat. No. 9,744,159, published US Patent Application No. 2017/348232 and WIPO International Patent Application WO2017015049, the contents of which are incorporated herein by reference in their entireties. Gaboxadol for the treatment of sleep apnea was disclosed in WO2005094820, the content of which is incorporated by reference herein in its entirety. Methods of treating depression with gaboxadol are disclosed in the published U.S. patent application No. 2009/0048288, the content of which is incorporated by reference herein in its entirety.

4) Human Equivalent Doses (HED)

Dosages of a medication administered to experimental animals, e.g. rodents, can be extrapolated to a human equivalent dose (HED) using the Body Surface Area (BSA) method (see, e.g. the “Guidance for Industry: Estimating the Maximum Safe Starting Dose in Initial Clinical Trials for Therapeutics in Adult Healthy Volunteers” (July 2005) available for download on the FDA's web site at www.fda.gov/files/drugs/published/Estimating-the-Maximum-Safe-Starting-Dose-in-Initial-Clinical-Trial s-for-Therapeutics-in-Adult-Healthy-Volunteers.pdf, the content of which is incorporated by reference herein in its entirety; see Table I below).

The human equivalent dose (HED) can be calculated using the following equation¹:

$$Formula for Dose Translation Based on B S A$$

$$H E D ( mg / kg ) = Animal dose ( mg / kg ) multiplied by \frac{Animal Km}{Human Km}$$

Hence, a daily human equivalent dose (mg/kg) or a daily dose (mg) administered to an adult human. human can be extrapolated from a daily dose of a medication administered to a mouse (mg/kg). ¹Kₘ refers to a conversion factor in kg/m² equal to the body weight in kg divided by the surface area in m²

TABLE I
Conversion of Animal Doses to Human Equivalent Doses Based on Body Surface Area

| | | | | | To Convert Animal Dose in mg/kg | |
|---|---|---|---|---|---|---|
| | | | | To Convert Dose in mg/kg to | to HED^{b} in mg/kg, Either | |
| | Reference | Working Weight | Body Surface | Dose in mg/m² | Divide | Multiply |
| Species | Body Weight | Range^{a} (kg) | Area (m²) | Multiply by kₘ | Animal Dose By | Animal Dose By |
| Human | 60 | — | 1.62 | 37 | — | — |
| Child^{c} | 20 | — | 0.80 | 25 | — | — |
| Mouse | 0.020 | 0.011-0.034 | 0.007 | 3 | 12.3 | 0.081 |
| Hamster | 0.080 | 0.047-0.157 | 0.016 | 5 | 7.4 | 0.135 |
| Rat | 0.150 | 0.080-0.270 | 0.025 | 6 | 6.2 | 0.162 |
| Ferret | 0.300 | 0.160-0.540 | 0.043 | 7 | 5.3 | 0.189 |
| Guinea pig | 0.400 | 0.208-0.700 | 0.05 | 8 | 4.6 | 0.216 |
| Rabbit | 1.8 | 0.9-3.0 | 0.15 | 12 | 3.1 | 0.324 |
| Dog | 10 | 5-17 | 0.50 | 20 | 1.8 | 0.544 |
| Primates: | | | | | | |
| Monkeys^{d} | 3 | 1.4-4.9 | 0.25 | 12 | 3.1 | 0.324 |
| Marmoset | 0.350 | 0.140-0.720 | 0.06 | 6 | 6.2 | 0.162 |
| Squirrel monkey | 0.600 | 0.200-0.970 | 0.09 | 7 | 5.3 | 0.189 |
| Baboon | 12 | 7-23 | 0.60 | 20 | 1.8 | 0.541 |
| Micro-pig | 20 | 10-33 | 0.74 | 27 | 1.4 | 0.730 |
| Mini-pig | 40 | 25-64 | 1.14 | 35 | 1.1 | 0.946 |

^{a}For animal weights within the specified ranges, the HED for a 60 kg human calculated using the standard kₘ, value will not vary more
than ±20 percent from the HED calculated using a kₘ value based on the exact animal weight.
^{b}Assumes 60 kg human. For species not listed or for weights outside the standard ranges, human equivalent dose can be calculated from the formula: HED = animal dose in mg/kg × (animal weight in kg/human weight in kg)^{0.33}.
^{c}The km value is provided for reference only since healthy children will rarely be volunteers for phase 1 trials.
^{d}For example, cynomolgus, rhesus, and stumptail.

a) Lithium

Unless otherwise indicated, as used herein, the term “lithium” refers to any lithium-containing compound including lithium salts, such as lithium carbonate, cocrystals as well as synthetic lithium pharmaceuticals, such as isotope-modified lithium compounds.

Lithium Salts

The most common lithium-containing compound for treatment of mental conditions is naturally occurring lithium carbonate (Li₂CO₃). The lithium carbonate molecule consists of a central carbon atom bonded to the oxygen ions, with two oxygen ions each bonded to a lithium ion. The electron valence of the constituent atoms dictates both the molecular structure and the chemical and biochemical reactions of the molecule. The molecular weight of Li being 6.94 g/mol and that of Li₂CO₃ mass being 73.89 g/mol, the mass of lithium ion (Li+) in a dose of lithium carbonate equates to 18.79% of the mass of lithium carbonate (Li₂CO₃).

In certain embodiments, other salt forms that can serve as a source of lithium include, but are not limited to, for example, lithium benzoate, lithium bromide, lithium fluoride, lithium cacodylate, lithium caffeine sulfonate, lithium chloride, lithium orotate, lithium citrate, lithium dithiosalicylate, lithium formate, lithium glycerophosphate, lithium iodate, lithium lactate and lithium salicylate. Lithium citrate (Li₃C₆H₅O₇) is approved by the FDA for treating mania and bipolar disorder and is available to be taken orally in the form of capsules, syrup and tablets. Lithium orotate (LiC₅H₃N₂O₄) and some other lithium compounds can be commercially available over the counter as vitamins.

In certain embodiments, a source of lithium does not comprise lithium gaboxadol salt.

In certain embodiments, a composition of a lithium salt, preferably an organic anion salt of lithium, and a complementary neutral organic compound are combined in a stoichiometric ratio. The cocrystal has the formula LiX.aM, wherein X is, for example, salicylate or lactate, M is a neutral organic molecule, and a is from 0.5 to 4. In specific variations of the invention, the lithium salicylate or lithium lactate has a molar ratio to the organic molecule of 1:1 or 1:2. Optionally, the organic molecule is an amino acid, synthetic amino acid, xanthine, polyphenol, or sugar. In general, organic anion lithium ionic cocrystal compositions may be prepared by combining the lithium salt and the complementary organic compound (i.e., the cocrystal precursor) in a solvent and using a commonly used method to promote crystallization such as evaporating or cooling the solvent to form the cocrystals.

Examples of amino acids or synthetic amino acids are alanine, arginine, asparagine, aspartic acid, cysteine, isoleucine, glutamic acid, glutamine, glycine, histidine, leucine, lysine, methionine, phenylalanine, proline, selenocysteine, serine, taurine, threonine, tryptophan, tyrosine, nicotinic acids, and valine. By way of further example, the amino acid is an L-amino acid such as L-phenylalanine, L-leucine, or L-tyrosine. In an alternative embodiment, the amino acid is a D-amino acid such as D-phenylalanine, D-leucine, or D-tyrosine. In an alternative embodiment, the cocrystal precursor comprises a non-proteinogenic amino acid. Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups; exemplary synthetic amino acids include β-amino acids and homo or β-analogs of natural (standard) amino acids. Other exemplary amino acids include pyrrolysine, betaine, and carnitine.

Examples of xanthine are caffeine, paraxanthine, theophylline, and threobromine.

Examples of polyphenols can be classified into the following categories: (1) phenolic acids, (2) flavonoids, (3) stilbenoids; (4) tannins, (5) monophenol such as hydroxytyrosol or p-tyrosol, (6) capsacin and other capsaicinoids and (7) curcumin. Phenolic acids form a diverse group including, for example, (a) hydroxycinnamic acids, e.g., p-coumaric acid, caffeic acid, and ferulic acid; (b) hydroxybenzoid acids, e.g., p-hydroxybenzoic acid, gallic acid, and ellagic acid; and (c) rosmarinic acid.

In certain embodiments, flavonoids can be resveratrol, epigallocatechin-3-gallate (EGCG), quercetin, ferulic acid, ellagic acid, hespereten, and protocatechuic acid.

In certain embodiments where the sugar as the organic molecule is a sugar, the sugar can include monosaccharides and disaccharides. For example, the sugar can be fructose, galactose, glucose, lactitol, lactose, maltitol, maltose, mannitol, melezitose, myoinositol, palatinite, raffinose, stachyose, sucrose, trehalose, or xylitol.

In certain embodiments, the composition optionally includes one or more nutraceuticals, selected from the group consisting of vitamin B2 (riboflavin), glucosamine HCl, chlorogenic acid, lipoic acid, catechin hydrate, creatine, acetyl-L-carnitine HCl, vitamin B6, pyridoxine, caffeic acid, naringenin, vitamin B1 (thiamine HCl), baicalein, luteolin, hesperedin, rosmarinic acid, epicatechin gallate, epigallocatechin, vitamin B9 (folic), genistein, methylvanillin, ethylvanillin, silibinin, diadzein, melatonin, rutin hydrate, vitamin A, retinol, vitamin D2 (ergocalciferol), vitamin E (tocopherol), diosmin, menadione (K3), vitamin D3 (caholecalciferol), phloretin, indole-3-carbinol, fisetin, glycitein, chrysin, gallocatechin, vitamin B4 (adenine), vitamin B5 (pantothenic acid), vitamin B7 (biotin), theobromine, resveratrol, epigallocatechin-3-gallate (EGCG), quercetin, ferulic acid, ellagic acid, hespereten, and protocatechuic acid. By way of further example, in this embodiment, the nutraceutical may be selected from the group consisting of vitamin B2 (riboflavin), glucosamine HCl, chlorogenic acid, lipoic acid, catechin hydrate, creatine, acetyl-L-carnitine HCl, vitamin B6, pyridoxine, caffeic acid, naringenin, vitamin B1 (thiamine HCl), baicalein, luteolin, hesperedin, rosmarinic acid, epicatechin gallate, epigallocatechin, vitamin B9 (folic), genistein, methylvanillin, ethylvanillin, silibinin, diadzein, melatonin, rutin hydrate, vitamin A, retinol, vitamin D2 (ergocalciferol), vitamin E (tocopherol), diosmin, menadione (K3), vitamin D3 (caholecalciferol), phloretin, indole-3-carbinol, fisetin, glycitein, chrysin, gallocatechin, vitamin B4 (adenine), vitamin B5 (pantothenic acid), vitamin B7 (biotin), theobromine, quercetin, ferulic acid, ellagic acid, hespereten, and protocatechuic acid.

In certain embodiments, the lithium salt and the complementary neutral organic compound are combined in an aqueous system. In certain embodiments, the lithium salt and complementary neutral organic compound may be dissolved in polar organic solvents such as acetone, acetonitrile, DMSO and alcohols.

In certain embodiments, organic anion lithium ionic cocrystal compositions may be prepared by combining a lithium-containing compound, an organic acid, and a complementary neutral organic compound, in a solvent, such as water, and using a commonly used method to promote crystallization such as evaporating or cooling the solvent.

Compositions and methods of making and administering lithium salt and/or cocrystal compounds are known in the art and are described, for example, in the U.S. Pat. No. 9,744,189, the content of which is incorporated by reference herein in its entirety.

### Isotope-Modified Lithium Compounds

Many atoms come in several stable isotopes, distinguished by the number of neutrons inside their atomic nucleus. Lithium has two stable isotopes, lithium-7 with 4 neutrons and lithium-6 with 3 neutrons. In nature, 92.5% of lithium atoms are lithium-7 while lithium-6 constitutes the other 7.5%. By and large, biology is insensitive to the different atomic isotopes. However, an experiment from 1986 reported that, while female mother rats fed lithium had “low” state of alertness compared to control rats fed a placebo, female rats fed lithium-6 had elevated alertness levels, reported as a “very high” state of alertness compared to the control rats. Therefore, synthetic lithium-6 purified compounds, comprised predominantly of lithium-6 (greater than 95% of the total lithium), may be effective at treating mental conditions with reduced alertness levels, such as chronic and major depression—disorders that are not well treated with the present lithium pharmaceuticals which all have the natural lithium-isotope abundance concentrations (i.e. 92.5% lithium-7 and only 7.3% lithium-6). Purifying lithium-6 requires synthetic means, since all naturally occurring lithium (as mined from dried lake beds, for example) contain the natural abundance of the lithium isotopes.

In naturally occurring lithium compounds, such as lithium carbonate, the concentration of lithium-7 and lithium-6 atoms matches nature's ratio-92.5% lithium-7 and 7.5% lithium-6. But, this concentration ratio can be modified by synthetic means, and synthetic isotope-modified lithium compounds may be used to treat various psychiatric disorders and conditions, including those resistant to existing medications or in combination with gaboxadol as disclosed herein.

### Li-6-Purified Compounds:

A purified Li-6 compound can be any lithium containing compound with lithium-6 present in an amount of at least 95% of the total lithium (i.e. lithium-6 and lithium-7) in the compound. The 95% threshold is much higher than the 7.5% natural abundance of lithium-6 that occurs in (un-synthesized) lithium pharmaceuticals, and is close to the ideal limit of lithium compounds with 100% lithium-6.

### Li-7-Purified Compounds:

A Li-7-purified compound can be any lithium containing compound with a percentage of lithium-7 in the compound being at least 99% of the total lithium content. This lithium-7 concentration is significantly higher than the 92.5% natural abundance of lithium-7. The Li-7-purified compounds can have very low lithium-6 concentrations (below 1%), much lower than the 7.5% natural lithium-6 abundance.

### Li-6-Enriched Compounds:

A Li-6-enriched compound can be any lithium containing compound with the percentage of lithium-6 present in the compound greater than 10% but less than 95% of the total lithium content. The 10% lithium-6 is significantly greater than the natural lithium-6 abundance of 7.5%. While lithium-6 concentrations in Li-6-enriched compounds can, in principle, be varied arbitrarily—in practical terms, it may be possible to control the concentration in increments of 10%. In certain embodiments, a Li-6-enriched compound class may comprise lithium-6 concentrations in the approximate ranges, about 10%-25%, about 25%-35%, about 35%-45%, about 45%-55%, about 55%-65%, about 65%-75%, about 75%-85% and about 85%-95%. In certain embodiments, the average lithium-6 concentrations in a Li-6-enriched compound can be about 20%, about 30%, about 40%, about 50%, about 60%, about 70%, about 80% and about 90%.

### Li-7-Enriched Compounds:

A Li-7-enriched compound can be any lithium containing compound having a percentage of lithium-7 greater than about 95% but less than about 99% of the total lithium content. The 95% lithium-7 is significantly greater than the natural lithium-7 abundance of 92.5%.

Methods of making and administering isotope-modified lithium compounds are known in the art and are described, for example, in the U.S. Pat. No. 9,044,418, the content of which is incorporated by reference herein in its entirety.

### Human Equivalent Doses of Lithium

In certain embodiments, the amount of lithium carbonate in mg/kg administered daily to a human adult can be extrapolated from a dosage. of lithium carbonate administered to an experimental animal, e.g. a mouse, using the aforementioned formula for dose translation based on BSA and by consulting, for example, TABLE II. In certain embodiments, a dosage of lithium carbonate refers to the amount of lithium carbonate in mg administered daily to a 60 adult human.

TABLE II
CONVERSION OF MOUSE LITHIUM CARBONATE (mg/kg)
DOSE TO HUMAN EQUIVALENT DOSE (HED) (mg/kg)
OF LITHIUM BASED ON BODY SURFACE AREA

| | | | | Daily dose for | |
|---|---|---|---|---|---|
| mg/kg | | mg/kg | | 60 kg human | |
| Li2CO3× | Animal Km | Li2CO3× | | | mg Li+ |
| MOUSE | Human Km= | HUMAN | 60 kg= | mg Li2CO3 | ion |
| 10× | 0.08= | 0.8× | 60= | 48 | 9.13 |
| 14.1× | 0.08= | 1.128× | 60= | 67.68 | 12.87 |
| 20× | 0.08= | 1.6× | 60= | 96 | 18.25 |
| 50× | 0.08= | 4× | 60= | 240 | 45.63 |
| 100× | 0.08= | 8× | 60= | 480 | 91.25 |
| 150× | 0.08= | 12× | 60= | 720 | 136.88 |
| 200× | 0.08= | 16× | 60= | 960 | 182.51 |
| 250× | 0.08= | 20× | 60= | 1200 | 228.14 |
| 300× | 0.08= | 24× | 60= | 1440 | 273.76 |
| 350× | 0.08= | 28× | 60= | 1680 | 319.39 |
| 400× | 0.08= | 32× | 60= | 1920 | 365.02 |
| 450× | 0.08= | 36× | 60= | 2160 | 410.65 |
| 500× | 0.08= | 40× | 60= | 2400 | 456.27 |

b) Gaboxadol

Unless otherwise indicated, as used herein, the term “gaboxadol” (e.g. Eskalith, Lithobid) refers to any gaboxadol-containing compound including gaboxadol salts, as well as, for example, deuterated and/or fluorinated pharmaceuticals.

Gaboxadol or THIP (4, 5, 6, 7-tetrahydroisoxazolo (5, 4-c) pyridin-3-ol) is a selective GABAA receptor agonist with a preference for δ-subunit containing GABAA receptors. Gaboxadol is described in EP Patent Nos. EP0000338, EP0840601, EP1641456, U.S. Pat. Nos. 4,278,676, 4,362,731, 4,353,910, and the published International Patent Application WO2005/094820, the contents of which are hereby incorporated by reference herein in their entireties.

### Gaboxadol Salts

Gaboxadol or pharmaceutically acceptable salt thereof may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In other suitable embodiments, inorganic acid addition salts, including but not limited to, hydrochloric, hydrobromic, sulfuric, sulfamic, phosphoric or nitric acid addition salts may be used. In certain embodiments, gaboxadol is provided as gaboxadol monohydrate.

In certain embodiments, base salt form of gaboxadol is prepared wherein the base is an inorganic base or an organic base selected from: aluminum, ammonium, calcium, copper, ferric, ferrous, magnesium, manganic salts, manganous, potassium, sodium, zinc bases, and primary, secondary, and tertiary amines, substituted amines including naturally occurring substituted amines, cyclic amines, and basic ion exchange resins, such as arginine, betaine, caffeine, choline, N,N-dibenzyl(ethylene)diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine.

In certain embodiments, the source of gaboxadol does not comprise lithium gaboxadol salt.

### Deuterated or Fluorinated Gaboxadol

In certain embodiments, gaboxadol is provided in deuterated or fluorinated form. One skilled in the art will readily understand that the amounts of active ingredient in a pharmaceutical composition will depend on the form of gaboxadol provided.

Deuteration and/or fluorination of pharmaceuticals to improve pharmacokinetics (PK), pharmacodynamics (PD), and toxicity profiles, has been demonstrated previously with some classes of drugs. Accordingly, the use of deuterium or fluorine enriched gaboxadol is contemplated and within the scope of the methods and compositions described herein. Deuterium or fluorine can be incorporated in any position in replacement of hydrogen synthetically, according to the synthetic procedures known in the art. For example, deuterium or fluorine may be incorporated to various positions having an exchangeable proton, such as the amine N—H, via proton-deuterium equilibrium exchange. Thus, deuterium or fluorine may be incorporated selectively or nonselectively through methods known in the art to provide deuterium enriched gaboxadol. See, for example, Journal of Labeled Compounds and Radiopharmaceuticals 19(5) 689-702 (1982).

Deuterium or fluorine enriched gaboxadol may be described by the percentage of incorporation of deuterium or fluorine at a given position in the molecule in the place of hydrogen. For example, deuterium enrichment of 1% at a given position means that 1% of molecules in a given sample contain deuterium at that specified position. The deuterium enrichment can be determined using conventional analytical methods, such as mass spectrometry and nuclear magnetic resonance spectroscopy. In embodiments deuterium enriched gaboxadol means that the specified position is enriched with deuterium above the naturally occurring distribution (i.e., above about 0.0156%). In embodiments deuterium enrichment is no less than about 1%, no less than about 5%, no less than about 10%, no less than about 20%, no less than about 50%, no less than about 70%, no less than about 80%, no less than about 90%, or no less than about 98% of deuterium at a specified position.

### Human Equivalent Dose of Gaboxadol

In certain embodiments, the amount of gaboxadol in mg/kg administered daily to a human adult can be extrapolated from a dosage of gaboxadol administered to an experimental animal, e.g. a mouse, using the aforementioned formula for dose translation based on BSA and by consulting, for example, TABLE III.

In certain embodiments, a dosage of gaboxadol refers to the amount of gaboxadol in mg administered daily to an adult human. In certain embodiments, gaboxadol is crystalline, such as the crystalline hydrochloric acid salt, the crystalline hydrobromic acid salt, or the crystalline zwitterion monohydrate. In certain embodiments, gaboxadol is provided as a crystalline monohydrate. In certain embodiments, 5.0, 10.0, 15.0, 33.0, 50.0 or 150.0 mg gaboxadol correspond to 5.6, 11.3, 16.9, 37, 56 or 169 mg gaboxadol monohydrate, respectively.

TABLE III
CONVERSION OF MOUSE GABOXADOL DOSE (mg/kg)
TO HUMAN EQUIVALENT DOSE (HED) OF GABOXADOL
(mg/kg) BASED ON BODY SURFACE AREA

| mg/kg | | mg/kg | DAILY DOSE | |
|---|---|---|---|---|
| gaboxadol× | Animal Km | gaboxadol× | (mg gaboxadol) | |
| (MOUSE) | Human Km= | (HUMAN) | for 60 kg HUMAN | |
| 1× | 0.08= | 0.08× | 60= | 4.8 |
| 3× | 0.08= | 0.24× | 60= | 14.4 |
| 6× | 0.08= | 0.48× | 60= | 28.8 |
| 10× | 0.08= | 0.8× | 60= | 48 |
| 15× | 0.08= | 1.2× | 60= | 72 |
| 20× | 0.08= | 1.6× | 60= | 96 |
| 25× | 0.08= | 2× | 60= | 120 |
| 30× | 0.08= | 2.4× | 60= | 144 |

5) Synergistic Combinations of Gaboxadol and Lithium

A proprietary and largely automated drug-screening platform called “pharmacomapping” comprises whole-brain detection of drug-evoked neuronal activation represented by drug-evoked expression of the immediate early genes (IEG), e.g., c-fos. Pharmacomapping is commercially available as a fee for service by CRO Certerra, Inc. in Farmingdale, N.Y.

Pharmacomapping of mouse or rat brain activity in response to various psychoactive medications, including antipsychotics, antidepressants, stimulants and anxiolytics (Engber et al., 1998; Salminen et al., 1996; SEMBA et al., 1996; Slattery et al., 2005; Sumner et al., 2004) confirmed that the imaging of c-fos activation in the rodent brain is a valid method of screening for psychoactive drugs (Sumner et al., 2004).

Using this experimental approach, low dose gaboxadol is shown in Examples 4 and 5A to act in synergy with a sub-standard dose of lithium to activate c-fos expression in the same regions of the brain as seen with standard therapeutically effective lithium monotherapy seen in Examples 2 and 3. Importantly, the brains of mice receiving either the low dose gaboxadol alone or sub-standard dose lithium alone did not elicit any detectable c-fos signaling activity. As a consequence, combination therapy using lower doses of gaboxadol and lithium, or a pharmaceutically acceptable salt of either or both compounds thereof, than normally used in monotherapy, may be effective, and side-effects associated with the larger amounts of lithium used in monotherapy may be reduced or prevented altogether.

Additionally, a standard dose of lithium also acts in synergy and/or additively with gaboxadol (Example 5B) suggesting that gaboxadol may be able to augment conventional lithium monotherapy especially in those patients who do not respond to conventional monotherapy or who relapse.

a) Sub-Standard Doses of Lithium

As used herein, a “sub-standard” dose of lithium defined as a human equivalent dose of lithium carbonate that when applied in an animal, such as a mouse, does not elicit any detectable or elicits only low activation of brain c-fos signaling.

In certain embodiments, a “sub-standard” dose of lithium is is defined as a daily dose of lithium carbonate that by itself, i.e. as lithium monotherapy, is unable to treat depression, treatment resistant depression acute suicidality or bipolar disorder.

In certain embodiments, a “sub-standard” dose of lithium in a human is a daily dose of less than about 10 mg lithium carbonate/kg, which corresponds to a dose of less than about 600 mg lithium carbonate/day, for an adult human.

In certain embodiments, a “sub-standard” daily dose of lithium for an adult human patient refers to a daily dose of about 50-600 mg lithium carbonate, about 55-600 mg lithium carbonate, about 60-600 mg lithium carbonate, about 65-600 mg lithium carbonate, about 70-600 mg lithium carbonate, about 75-600 mg lithium carbonate, about 80-600 mg lithium carbonate, about 85-600 mg lithium carbonate, about 90-600 mg lithium carbonate or about 95-600 mg lithium carbonate, about 100-600 mg lithium carbonate, about 105-600 mg lithium carbonate, about 110-600 mg lithium carbonate, about 115-100 mg lithium carbonate, about 120-100 mg lithium carbonate, about 125-600 mg lithium carbonate, about 130-600 mg lithium carbonate, about 135-600 mg lithium carbonate, about 140-600 mg lithium carbonate, about 145-600 mg lithium carbonate, about 150-600 mg lithium carbonate, about 155-600 mg lithium carbonate, about 160-600 mg lithium carbonate, about 165-600 mg lithium carbonate, about 170-600 mg lithium carbonate, about 175-600 mg lithium carbonate, about 180-600 mg lithium carbonate, about 185-600 mg lithium carbonate, about 190-600 mg lithium carbonate, about 195-600 mg lithium carbonate, about 200-600 mg lithium carbonate, about 215-600 mg lithium carbonate, about 210-600 mg lithium carbonate, about 215-100 mg lithium carbonate, about 220-100 mg lithium carbonate, about 225-600 mg lithium carbonate, about 230-600 mg lithium carbonate, about 235-600 mg lithium carbonate, about 240-600 mg lithium carbonate, about 245-600 mg lithium carbonate, about 250-600 mg lithium carbonate, about 255-600 mg lithium carbonate, about 260-600 mg lithium carbonate, about 265-600 mg lithium carbonate, about 270-600 mg lithium carbonate, about 275-600 mg lithium carbonate, about 280-600 mg lithium carbonate, about 285-600 mg lithium carbonate, about 290-600 mg lithium carbonate, about 295-600 mg lithium carbonate, about 300-600 mg lithium carbonate, about 315-600 mg lithium carbonate, about 310-600 mg lithium carbonate, about 315-600 mg lithium carbonate, about 320-600 mg lithium carbonate, about 325-600 mg lithium carbonate, about 330-600 mg lithium carbonate, about 335-600 mg lithium carbonate, about 340-600 mg lithium carbonate, about 345-600 mg lithium carbonate, about 350-600 mg lithium carbonate, about 355-600 mg lithium carbonate, about 360-600 mg lithium carbonate, about 365-600 mg lithium carbonate, about 370-600 mg lithium carbonate, about 375-600 mg lithium carbonate, about 380-600 mg lithium carbonate, about 385-600 mg lithium carbonate, about 390-600 mg lithium carbonate or about 395-600 mg lithium carbonate, inclusive of all values and ranges there between.

In certain embodiments, a “sub-standard” daily dose of lithium for an adult human adult patient refers to a daily dose of about 50-600 mg lithium carbonate, 50-595 mg lithium carbonate, 50-590 mg lithium carbonate, 50-585 mg lithium carbonate, 50-580 mg lithium carbonate, 50-575 mg lithium carbonate, 50-570 mg lithium carbonate, 50-565 mg lithium carbonate, 50-560 mg lithium carbonate, 50-555 mg lithium carbonate, 50-550 mg lithium carbonate, 50-545 mg lithium carbonate, 50-540 mg lithium carbonate, 50-535 mg lithium carbonate, 50-530 mg lithium carbonate, 50-525 mg lithium carbonate, 50-520 mg lithium carbonate, 50-515 mg lithium carbonate, 50-510 mg lithium carbonate, 50-505 mg lithium carbonate, 50-500 mg lithium carbonate, 50-495 mg lithium carbonate, 50-490 mg lithium carbonate, 50-485 mg lithium carbonate, 50-480 mg lithium carbonate, 50-475 mg lithium carbonate, 50-470 mg lithium carbonate, 50-465 mg lithium carbonate, 50-460 mg lithium carbonate, 50-455 mg lithium carbonate, 50-450 mg lithium carbonate, 50-445 mg lithium carbonate, 50-440 mg lithium carbonate, 50-435 mg lithium carbonate, 50-430 mg lithium carbonate, 50-425 mg lithium carbonate, 50-420 mg lithium carbonate, 50-415 mg lithium carbonate, 50-410 mg lithium carbonate, 50-405 mg lithium carbonate, 50-400 mg lithium carbonate, about 50-395 mg lithium carbonate, about 50-390 mg lithium carbonate, about 50-385 mg lithium carbonate, about 50-380 mg lithium carbonate, about 50-375 mg lithium carbonate, about 50-370 mg lithium carbonate, about 50-365 mg lithium carbonate, about 50-360 mg lithium carbonate, about 50-355 mg lithium carbonate, about 50-350 mg lithium carbonate, about 50-345 mg lithium carbonate, about 50-340 mg lithium carbonate, about 50-335 mg lithium carbonate, about 50-330 mg lithium carbonate, about 50-325 mg lithium carbonate, about 50-320 mg lithium carbonate, about 50-315 mg lithium carbonate, about 50-3500 mg lithium carbonate, about 50-305 mg lithium carbonate, about 50-300 mg lithium carbonate, about 50-300 mg lithium carbonate, about 50-295 mg lithium carbonate, about 50-290 mg lithium carbonate, about 50-285 mg lithium carbonate, about 50-280 mg lithium carbonate, about 50-275 mg lithium carbonate, about 50-270 mg lithium carbonate, about 50-265 mg lithium carbonate, about 50-260 mg lithium carbonate, about 50-255 mg lithium carbonate, about 50-250 mg lithium carbonate, about 50-245 mg lithium carbonate, about 50-240 mg lithium carbonate, about 50-235 mg lithium carbonate, about 50-230 mg lithium carbonate, about 50-225 mg lithium carbonate, about 50-220 mg lithium carbonate, about 50-215 mg lithium carbonate, about 50-210 mg lithium carbonate, about 50-205 mg lithium carbonate, about 50-200 mg lithium carbonate, about 50-195 mg lithium carbonate, about 50-190 mg lithium carbonate, about 50-185 mg lithium carbonate, about 50-180 mg lithium carbonate, about 50-175 mg lithium carbonate, about 50-170 mg lithium carbonate, about 50-165 mg lithium carbonate, about 50-160 mg lithium carbonate, about 50-155 mg lithium carbonate, about 50-150 mg lithium carbonate, about 50-145 mg lithium carbonate, about 50-140 mg lithium carbonate, about 50-135 mg lithium carbonate, about 50-130 mg lithium carbonate, about 50-125 mg lithium carbonate, about 50-120 mg lithium carbonate, about 50-115 mg lithium carbonate, about 50-110 mg lithium carbonate, about 50-105 mg lithium carbonate, about 50-100 mg lithium carbonate, about 50-95 mg lithium carbonate, about 50-90 mg lithium carbonate, about 50-85 mg lithium carbonate, about 50-80 mg lithium carbonate, about 50-75 mg lithium carbonate, about 50-70 mg lithium carbonate, about 50-65 mg lithium carbonate, about 50-60 mg lithium carbonate, about 50-55 mg lithium carbonate inclusive of all values and ranges there between.

b) Standard Doses of Lithium

As used herein, a “standard” dose of lithium is defined as a daily dose of lithium that by itself, i.e. as a single dose, elicits brain c-fos signaling in an animal model, such as a mouse.

In certain embodiments, a “standard” dose of lithium is defined as a daily dose of lithium that by itself, i.e. as lithium monotherapy, can treat depression, treatment resistant depression acute suicidality or bipolar disorder.

In certain embodiments, a “standard” dose of lithium in a human is a daily dose of more than about 10 mg/kg of lithium carbonate, which corresponds to a dose of more than about 600 mg lithium carbonate/day for an adult human.

In certain embodiments, a “standard” dose of lithium in mice is a daily dose in the range between about 120 mg/kg and 480 mg/kg of lithium carbonate. The human equivalent dose corresponds to about 10 mg/kg and 40 mg/kg, which, for an adult human, is a dose from about 600 mg to about 2400 mg lithium carbonate/day.

In certain embodiments, a “standard” daily dose of lithium for an adult human patient refers to a daily dose of about 600-2400 mg lithium carbonate,

In certain embodiments, a “standard” daily dose of lithium for an adult human patient refers to a daily dose of about 600-2400 mg lithium carbonate,

In certain embodiments, a “standard” daily dose of lithium for an adult human patient refers to a daily dose of about 600-2350 mg lithium carbonate, about 600-2300 mg lithium carbonate, about 600-2250 mg lithium carbonate, about 600-2200 mg lithium carbonate, about 600-2150 mg lithium carbonate, about 600-2100 mg lithium carbonate, about 600-2050 mg lithium carbonate, about 600-2000 mg lithium carbonate, about 600-1950 mg lithium carbonate, about 600-1900 mg lithium carbonate, about 600-1850 mg lithium carbonate, about 600-1800 mg lithium carbonate, about 600-1750 mg lithium carbonate, about 600-1700 mg lithium carbonate, about 600-1650 mg lithium carbonate, about 600-1600 mg lithium carbonate, about 600-1550 mg lithium carbonate, about 600-1500 mg lithium carbonate, about 600-1450 mg lithium carbonate, about 600-1400 mg lithium carbonate, about 600-1350 mg lithium carbonate, about 600-1300 mg lithium carbonate, about 600-1250 mg lithium carbonate, about 600-1200 mg lithium carbonate, about 600-1150 mg lithium carbonate, about 600-1100 mg lithium carbonate, about 600-1050 mg lithium carbonate, about 600-1000 mg lithium carbonate, about 600-950 mg lithium carbonate, about 600-900 mg lithium carbonate, about 600-850 mg lithium carbonate, about 600-800 mg lithium carbonate, about 600-750 mg lithium carbonate, about 600-700 mg lithium carbonate, or about 600-650 mg lithium carbonate inclusive of all values and ranges there between.

In certain embodiments, a “standard” daily dose of lithium for an adult human patient refers to a daily dose of about 600-2400 mg lithium carbonate, about 650-2400 mg lithium carbonate, about 700-2400 mg lithium carbonate, about 750-2400 mg lithium carbonate, about 800-2400 mg lithium carbonate, about 850-2400 mg lithium carbonate, about 900-2400 mg lithium carbonate, about 950-2400 mg lithium carbonate, about 1000-2400 mg lithium carbonate, about 1050-2400 mg lithium carbonate, about 1050-2400 mg lithium carbonate, about 1100-2400 mg lithium carbonate, about 1150-2400 mg lithium carbonate, about 1200-2400 mg lithium carbonate, about 1250-2400 mg lithium carbonate, about 1300-2400 mg lithium carbonate, about 1350-2400 mg lithium carbonate, about 1400-2400 mg lithium carbonate, about 1450-2400 mg lithium carbonate, about 1500-2400 mg lithium carbonate, about 1550-2400 mg lithium carbonate, about 1600-2400 mg lithium carbonate, about 1650-2400 mg lithium carbonate, about 1700-2400 mg lithium carbonate, about 1750-2400 mg lithium carbonate, about 1800-2400 mg lithium carbonate, about 1850-2400 mg lithium carbonate, about 1900-2400 mg lithium carbonate, about 1950-2400 mg lithium carbonate, about 2000-2400 mg lithium carbonate, about 2050-2400 mg lithium carbonate, about 2100-2400 mg lithium carbonate, about 2150-2400 mg lithium carbonate, about 2200-2400 mg lithium carbonate, about 2250-2400 mg lithium carbonate, about 2300-2400 mg lithium carbonate or about 2350-2400 mg inclusive of all values and ranges there between.

c) Low to Medium Doses of Gaboxadol

As used herein, a “low” daily dose of gaboxadol is defined as a dose of gaboxadol, that by itself, does not elicit any activation of detectable brain c-fos signaling in animal model testing, while medium dose of gaboxadol is defined as a dose of gaboxadol, that by itself, elicits only modest activation of detectable brain c-fos signaling in animal model testing.

In certain embodiments, a “low dose” dose of gaboxadol is defined in mice as a single dose between 1 and 3 mg/kg, corresponding to human equivalent dose of about between 5 to 15 mg for an adult human.

In certain embodiments, a “medium dose” dose of gaboxadol in mice is a single dose between 3 and 6 mg/kg, corresponding to human equivalent dose of about between 15 to 30 mg for an adult human.

In certain embodiments, a “low” to “medium” dose of gaboxadol in mice is a daily dose in the range of about 1 to about 6 mg/kg. The human equivalent dose equates to about 0.081 to about 0.49 mg/kg, which corresponds to a dose in a range from about 5 to about 30 mg gaboxadol/day, for an adult human.

In certain embodiments, a “low” dose of gaboxadol for an adult human patient refers to a daily dose of about 5 to about 15 mg gaboxadol, about 5 to about 18 mg gaboxadol, about 5 to about 17 mg gaboxadol, about 5 to about 16 mg gaboxadol, about 5 to about 15 mg gaboxadol, about 5 to about 14 mg gaboxadol, about 5 to about 13 mg gaboxadol, about 5 to about 12 mg gaboxadol, about 5 to about 11 mg gaboxadol, about 5 to about 10 mg gaboxadol, about 5 to about 9 mg gaboxadol, about 5 to about 8 mg gaboxadol, about 5 to about 7 mg gaboxadol, about 5 to about 6 mg gaboxadol, inclusive of all values and ranges there between.

In certain embodiments, a “low” dose of gaboxadol for an adult human patient refers to a daily dose of about 5 to about 15 mg gaboxadol, about 6 to about 15 mg gaboxadol, about 7 to about 15 mg gaboxadol, about 8 to about 15 mg gaboxadol, about 9 to about 15 mg gaboxadol, about 10 to about 15 mg gaboxadol, about 11 to about 15 mg gaboxadol, about 12 to about 15 mg gaboxadol, about 13 to about 15 mg gaboxadol, about 14 to about 15 mg gaboxadol, inclusive of all values and ranges there between.

In certain embodiments, a “medium” dose of gaboxadol in mice is a daily dose in the range of about 3 to about 6 mg/kg. The human equivalent dose equates to about 0.24 to about 0.48 mg/kg, which corresponds to a dose in a range from about 15 to about 30 mg gaboxadol/day, for an adult human.

In certain embodiments, a “medium” dose of gaboxadol for an adult human patient refers to a daily dose of about 15 to about 30 mg gaboxadol, about 16 to about 30 mg gaboxadol, about 17 to about 30 mg gaboxadol, about 18 to about 30 mg gaboxadol, about 19 to about 30 mg gaboxadol, about 20 to about 30 mg gaboxadol, about 21 to about 30 mg gaboxadol, about 22 to about 30 mg gaboxadol, about 23 to about 30 mg gaboxadol, about 24 to about 30 mg gaboxadol, about 25 to about 30 mg gaboxadol, about 26 to about 30 mg gaboxadol, about 27 to about 30 mg gaboxadol, about 28 to about 30 mg gaboxadol or about 29 to about 30 mg gaboxadol, inclusive of all values and ranges there between.

In certain embodiments, a “medium” dose of gaboxadol for an adult human patient refers to a daily dose of about 15 to about 30 mg gaboxadol, about 15 to about 29 mg gaboxadol, about 15 to about 28 mg gaboxadol, about 15 to about 27 mg gaboxadol, about 0.5 to about 26 mg gaboxadol, about 15 to about 25 mg gaboxadol, about 15 to about 24 mg gaboxadol, about 0.5 to about 23 mg gaboxadol, about 15 to about 22 mg gaboxadol, about 15 to about 21 mg gaboxadol, about 15 to about 20 mg gaboxadol, about 15 to about 19 mg gaboxadol, about 15 to about 18 mg gaboxadol, about 15 to about 17 mg gaboxadol or about 15 to about 16 mg gaboxadol inclusive of all values and ranges there between.

d) High Doses of Gaboxadol

As used herein, a “high” daily dose of gaboxadol is defined as a dose of gaboxadol that by itself strongly increases c-fos signaling in the brain in an animal model testing.

As used herein, a “high” dose of gaboxadol is a daily dose in mice of 6-30 mg/kg of gaboxadol. The human equivalent dose equates to about 0.48 to about 2.4 mg/kg, which corresponds to a dose in a range from about 30 to about 150 mg gaboxadol/day, for an adult human.

In certain embodiments, a “high” dose of gaboxadol for an adult human refers to a daily dose of 30-150 mg gaboxadol, about 35-150 mg gaboxadol, about 40-150 mg gaboxadol, about 45-150 mg gaboxadol, about 50-150 mg gaboxadol, about 55-150 mg gaboxadol, about 60-150 mg gaboxadol, about 65-150 mg gaboxadol, about 70-150 mg gaboxadol, about 75-150 mg gaboxadol, about 80-150 mg gaboxadol, about 85-150 mg gaboxadol, about 90-150 mg gaboxadol, about 95-150 mg gaboxadol, about 100-150 mg gaboxadol, about 105-150 mg gaboxadol, about 110-150 mg gaboxadol, about 115-150 mg gaboxadol, about 120-150 mg gaboxadol, about 125-150 mg gaboxadol, about 130-150 mg gaboxadol, about 135-150 mg gaboxadol, about 140-150 mg gaboxadol, about 145-150 mg gaboxadol, inclusive of all values and ranges there between.

In certain embodiments, a “high” dose of gaboxadol for an adult human refers to a daily dose of about 30-300 mg gaboxadol, about 30-245 mg gaboxadol, about 30-240 mg gaboxadol, about 30-235 mg gaboxadol, about 30-230 mg gaboxadol, about 30-230 mg gaboxadol, about 30-220 mg gaboxadol, about 30-215 mg gaboxadol, about 30-210 mg gaboxadol, about 30-205 mg gaboxadol, about 30-200 mg gaboxadol, about 30-195 mg gaboxadol, about 30-190 mg gaboxadol, about 30-185 mg gaboxadol, about 30-180 mg gaboxadol, about 30-175 mg gaboxadol, about 30-170 mg gaboxadol, about 30-165 mg gaboxadol, about 30-160 mg gaboxadol, about 30-155 mg gaboxadol, about 30-150 mg gaboxadol, about 30-145 mg gaboxadol, about 30-140 mg gaboxadol, about 30-135 mg gaboxadol, about 30-130 mg gaboxadol, about 30-130 mg gaboxadol, about 30-120 mg gaboxadol, about 30-115 mg gaboxadol, about 30-110 mg gaboxadol, about 30-105 mg gaboxadol, about 30-100 mg gaboxadol, about 30-95 mg gaboxadol, about 30-90 mg gaboxadol, about 30-85 mg gaboxadol, about 30-80 mg gaboxadol, about 30-75 mg gaboxadol, about 30-70 mg gaboxadol, about 30-65 mg gaboxadol, about 30-60 mg gaboxadol, about 30-55 mg gaboxadol, about 30-50 mg gaboxadol, about 30-45 mg gaboxadol, about 30-40 mg gaboxadol or about 30-35 mg gaboxadol inclusive of all values and ranges there between.

e) Synergistic Combinations of Gaboxadol and Lithium

In many embodiments, the effective amounts of lithium and gaboxadol are synergistic amounts. As used herein, a “synergistic combination” or a “synergistic amount” of lithium and gaboxadol is a combined dosage that is more effective in the therapeutic or prophylactic treatment of a psychiatric disorder than the incremental improvement in treatment outcome that could be predicted or expected from a merely additive combination of (i) the therapeutic or prophylactic benefit of lithium when administered at that same dosage as a monotherapy and (ii) the therapeutic or prophylactic benefit of gaboxadol when administered at the same dosage as monotherapy.

In certain embodiments, the administration of a “synergistic combination” of lithium and gaboxadol to a subject in need thereof activates c-fos signaling in at least one region of the animal model's brain selected from the group consisting of: 1) a broad cortical activation comprising motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, claustrum (CLA), as well as 2) subcortical activation comprising hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS).

In certain embodiments, the administration of a “synergistic combination” of lithium and gaboxadol to a subject in need thereof activates c-fos signaling in at least two regions of the animal model's brain selected from the group consisting of 1) a broad cortical activation comprising motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, claustrum (CLA), as well as 2) subcortical activation comprising hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS).

In certain embodiments, the administration of a “synergistic combination” of lithium and gaboxadol to a subject in need thereof activates c-fos signaling in at least three regions of an animal model's brain selected from the group consisting of 1) a broad cortical activation comprising motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, claustrum (CLA), as well as 2) subcortical activation comprising hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS).

In certain embodiments, the administration of a “synergistic combination” of lithium and gaboxadol to a subject in need thereof activates c-fos signaling in 1) a broad cortical activation comprising motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, claustrum (CLA), as well as 2) subcortical activation comprising hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS) of the animal model's brain.

In certain embodiments, the administration of a “synergistic combination” of lithium and gaboxadol to a subject in need thereof activates c-fos signaling in at least one, two, three or more regions of the animal model's brain that are also activated by lithium monotherapy.

In certain embodiments, the administration of a “synergistic combination” of lithium and gaboxadol to a subject in need thereof activates c-fos signaling in at least one, two, three or more regions of the animal model's brain that are also activated by gaboxadol monotherapy.

In certain embodiments, the “synergistic combination” of lithium and gaboxadol comprises a sub-standard dose of lithium as defined herein.

In certain embodiments, the “synergistic combination” of lithium and gaboxadol comprises a standard dose of lithium as defined herein.

In certain embodiments, the “synergistic combination” of lithium and gaboxadol comprises a low dose of gaboxadol as defined herein.

In certain embodiments, the “synergistic combination” of lithium and gaboxadol comprises a medium dose of gaboxadol as defined herein.

In certain embodiments, the “synergistic combination” of lithium and gaboxadol comprises a high dose of gaboxadol as defined herein.

In certain embodiments, a sub-standard dose of lithium as defined herein is administered with a low dose of gaboxadol as exemplified in Example 4.

In certain embodiments, the “synergistic combination” of lithium and gaboxadol comprises a daily dose for an adult human patient of about 50-600 mg lithium carbonate and about 5 to about 30 mg gaboxadol.

In certain embodiments, the “synergistic combination” of lithium and gaboxadol comprises a daily dose for an adult human patient of about 50-600 mg lithium carbonate and about 30 to about 150 mg gaboxadol.

In certain embodiments, the “synergistic combination” of lithium and gaboxadol comprises a daily dose for an adult human patient of about 600 mg to about 2400 mg lithium carbonate and about 5 to about 30 mg gaboxadol.

In certain embodiments, the “synergistic combination” of lithium and gaboxadol comprises a daily dose for an adult human patient of about 600 mg to about 2400 mg lithium carbonate and about 30 to about 150 mg gaboxadol.

In certain embodiments, the “synergistic combination” of lithium and gaboxadol does not comprise lithium and gaboxadol at a molar ratio of 1:1.

In certain embodiments, synergy between lithium and gaboxadol results in an activation of an immediate early gene (e.g. c-fos, arc, egr-1, fosb and npas4) in the brain of an animal model that is at least about 5%, 10%, 20%, 30%, 40%, 50%, 100%, 200%, 400%, 500% or 1000% greater than the sum of the effects of lithium alone and gaboxadol alone on brain c-fos signaling (additive effect).

In certain embodiments, synergy between lithium and gaboxadol results in an activation of of c-fos gene expression in the brain of an animal model that is at least about 5%, 10%, 20%, 30%, 40%, 50%, 100%, 200%, 400%, 500% or 1000% greater than the sum of the effects of lithium alone and gaboxadol alone on brain c-fos signaling (additive effect).

In certain embodiments, the additive effect between lithium and gaboxadol results in an activation of an immediate early gene (e.g. c-fos, arc, egr-1, fosb and npas4) in the brain of an animal model that is equal to the sum of the effects of lithium alone and gaboxadol alone on brain c-fos signaling.

In certain embodiments, the additive effect between lithium and gaboxadol results in an activation of the immediate early c-fos gene in the brain of an animal model that is equal to the sum of the effects of lithium alone and gaboxadol alone on brain c-fos signaling.

In certain embodiments, an “additive combination” of lithium and gaboxadol comprises a sub-standard dose of lithium as defined herein.

In certain embodiments, an “additive combination” of lithium and gaboxadol comprises a standard dose of lithium as defined herein.

In certain embodiments, an “additive combination” of lithium and gaboxadol comprises a low dose of gaboxadol as defined herein.

In certain embodiments, an “additive combination” of lithium and gaboxadol comprises a medium dose of gaboxadol as defined herein.

In certain embodiments, an “additive combination” of lithium and gaboxadol comprises a high dose of gaboxadol as defined herein.

In certain embodiments, an “additive combination” of lithium and gaboxadol does not comprise lithium and gaboxadol at a molar ratio of 1:1.

In certain embodiments, lithium and gaboxadol can be administered as separate compositions for combination therapy (which indicates that they are formulated separately) or together (which indicates that they are formulated together).

In certain embodiments, lithium and gaboxadol can be administered simultaneously or contemporaneously as defined herein.

6) Treatment of Psychiatric Disorders with a Synergistic Combination of Gaboxadol and Lithium

Based on the similarities of the pharmacomaps between the gaboxadol and lithium synergistic combination therapy and that seen with conventional lithium monotherapy, gaboxadol augments the established activity of lithium in the treatment of bipolar disorder, depression, treatment resistant depression and acute suicidality (see above).

Thus, in certain embodiments, a method of treating bipolar disorder, depression, treatment-resistant depression and acute suicidality is disclosed comprising administering a synergistic combination of gaboxadol and lithium, or a pharmaceutically acceptable salt of either or both compounds thereof, to a patient in need thereof.

In certain embodiments, the efficacy of the treatment can be monitored by a physician using a disease-specific psychiatric rating scale. A psychiatric rating scale refers to a psychological test that has been developed to provide a reliable and objective method of monitoring symptom severity of a particular mood disorder and measuring a response to treatment, see, e.g., Handbook of clinical rating scales and assessment in psychiatry and mental health by Baer, Lee and Blais, Mark A. New York; Humana Press, 2010; ISBN: 9781588299666, the content of which is incorporated by reference herein in its entirety.

Exemplary psychiatric rating scales include, but are not limited to,
Beck Depression Inventory (BDI), Beck Hopelessness Scale, Centre for Epidemiological Studies—Depression Scale (CES-D), Center for Epidemiological Studies Depression Scale for Children (CES-DC), Edinburgh Postnatal Depression Scale (EPDS), Geriatric Depression Scale (GDS), Hamilton Rating Scale for Depression (HAM-D), Hospital Anxiety and Depression Scale, Kutcher Adolescent Depression Scale (KADS), Major Depression Inventory (MDI), Montgomery-Asberg Depression Rating Scale (MADRS), PHQ-9, Mood and Feelings Questionnaire (MFQ), Weinberg Screen Affective Scale (WSAS) and Zung Self-Rating Depression Scale for depression;
Altman Self-Rating Mania Scale (ASRM), Bipolar Spectrum Diagnostic Scale, Child Mania Rating Scale, General Behavior Inventory, Hypomania Checklist, Mood Disorder Questionnaire (MDQ), Young Mania Rating Scale (YMRS) for mania and bipolar disorder
SAD PERSONS scale for suicide risk.

In certain embodiments, the efficacy of the treatment can be monitored by a physician using a EEG recordings during and in the period after the drug combination application, with drug-evoked biomarker changes in EEG based on drug-evoked biomarker changes established preclinically in animal models.

7) Formulations of Gaboxadol and Lithium

Methods of administration of the synergistic combination of gaboxadol and lithium, or a pharmaceutically acceptable salt of either or both compounds thereof, include but are not limited to, oral, subcutaneous, intradermal, intramuscular (by way of non-limiting example, intramuscular depot, such as, for instance, as described in U.S. Pat. No. 6,569,449, the content of which are hereby incorporated by reference in its entirety), intraperitoneal, intravenous, intranasal, epidural, sublingual, intranasal, intracerebral, intravaginal, transdermal, rectally, by inhalation, or topically, particularly to the ears, nose, eyes, or skin. The mode of administration can be left to the discretion of the practitioner. In most instances, administration results in the release of the compounds described herein or their pharmaceutically acceptable salts into the bloodstream.

In certain embodiments, the invention contemplates administration of gaboxadol and lithium, or a pharmaceutically acceptable salt of either or both compounds thereof, designed for rapid onset of treatment effect. A wide variety of dose forms may be employed including those described previously in the literature. Preferred dose forms are suitable for oral or intranasal administration. Compositions for oral delivery can be in the form of tablets, lozenges, aqueous or oily suspensions, solutions, granules, capsules, powders, pills, pellets, capsules containing liquids, emulsions, syrups, or elixirs, suppositories, sustained-release formulations, or any other form suitable for use.

Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active compound of the invention are also suitable for oral administration. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

The present compositions can optionally comprise a suitable amount of a pharmaceutically acceptable excipient so as to provide the form for proper administration to the subject. Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when the compounds of present invention or their pharmaceutically acceptable salts are administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, sodium chloride, dried skim milk, glycerol, propylene, glycol, water, ethanol and the like. The present compositions, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference in its entirety.

A particularly preferred form for rapid onset is an orally disintegrating dosage form (ODDF) which provides immediate release in the patient's buccal cavity enhancing buccal absorption of the drug. An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets as is common with conditions which are psychiatric in nature.

In certain embodiments, pharmaceutical compositions herein provide immediate release of gaboxadol and lithium, or a pharmaceutically acceptable salt of either or both compounds thereof, which when administered to an oral cavity, disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds based upon, e.g., the United States Pharmacopeia (USP) disintegration test method set forth at section 701, Revision Bulletin Official Aug. 1, 2008.

In preferred embodiments, the ODDF results in pharmacokinetic properties which include a Tmax of 20 minutes or less. In certain embodiments, pharmaceutical compositions herein provide of 20 minutes or less, a Tmax of 19 minutes or less, a Tmax of 18 minutes or less, a Tmax of 17 minutes or less, a Tmax of 16 minutes or less, a Tmax of 15 minutes or less, a Tmax of 14 minutes or less, a Tmax of 13 minutes or less, a Tmax of 12 minutes or less, a Tmax of 11 minutes or less, a Tmax of 10 minutes or less, a Tmax of 9 minutes or less, a Tmax of 8 minutes or less, a Tmax of 7 minutes or less, a Tmax of 6 minutes or less, or a Tmax of 5 minutes or less. Such pharmaceutical compositions include ODDFs such as orally disintegrating tablets (ODTs).

An ODT is a solid dosage form containing a medicinal substance or active ingredient which disintegrates rapidly, usually within a matter of seconds when placed upon the tongue. The disintegration time for ODTs generally ranges from several seconds to about a minute. ODTs are designed to disintegrate or dissolve rapidly on contact with saliva, thus eliminating the need to chew the tablet, swallow the intact tablet, or take the tablet with liquids. As with ODDFs in general, this mode of administration can be beneficial to people who require rapid onset of treatment.

In certain embodiments, the fast dissolving property of the ODTs requires quick ingress of water into the tablet matrix. This may be accomplished by maximizing the porous structure of the tablet, incorporation of suitable disintegrating agents and use of highly water-soluble excipients in the formulation. Excipients used in ODTs typically contain at least one superdisintegrant (which can have a mechanism of wicking, swelling or both), a diluent, a lubricant and optionally a swelling agent, sweeteners and flavorings. See, e.g., Nagar et al., Journal of Applied Pharmaceutical Science, 2011; 01 (04):35-45. Superdisintegrants can be classified as synthetic, natural and co-processed. In this context synthetic superdisintegrants can be exemplified by sodium starch glycolate, croscarmellose sodium, cross-linked polyvinylpyrrolidone, low-substituted hydroxypropyl cellulose, microcrystalline cellulose, partially pregelatinized starch, cross-linked alginic acid and modified resin. Natural superdisintegrants can be processed mucilages and gums are obtained from plants and can be exemplified by Lepidium sativum seed mucilage, banana powder, gellan gum, locust bean gum, xanthan gum, guar gum, gum karaya, cassia fistula seed gum, Mangifera indica gum, carrageenan, agar from Gelidium amansii and other red algaes, soy polysaccharide and chitosan. Diluents can include, e.g., mannitol, sorbitol, xylitol, calcium carbonate, magnesium carbonate, calcium sulfate, magnesium trisilicate and the like. Lubricants can include, e.g., magnesium stearate and the like. Those skilled in the art are familiar with ODT manufacturing techniques.

Other ODDFs which may be used herein include rapidly dissolving films which are thin oral strips that release medication such as gaboxadol and lithium, or a pharmaceutically acceptable salt of either or both compounds thereof, quickly after administration to the oral cavity. The film is placed on a patient's tongue or any other mucosal surface and is instantly wet by saliva whereupon the film rapidly hydrates and dissolves to release the medication. See. e.g., Chaturvedi et al., Curr Drug Deliv. 2011 July; 8 (4):373-80. Fastcaps are a rapidly disintegrating drug delivery system based on gelatin capsules. In contrast to conventional hard gelatin capsules, fastcaps consist of a gelation of low bloom strength and various additives to improve the mechanical and dissolution properties of the capsule shell. See, e.g., Ciper and Bodmeier, Int J Pharm. 2005 Oct. 13; 303 (1-2):62-71. Freeze dried (lyophilized) wafers are rapidly disintegrating, thin matrixes that contain a medicinal agent. The wafer or film disintegrates rapidly in the oral cavity and releases drug which dissolves or disperses in the saliva. See, e.g., Boateng et al., Int J Pharm. 2010 Apr. 15; 389 (1-2):24-31. Those skilled in the art are familiar with various techniques utilized to manufacture ODDFs such as freeze drying, spray drying, phase transition processing, melt granulation, sublimation, mass extrusion, cotton candy processing, direct compression, etc. See, e.g., Nagar et al., supra.

When administered, ODDFs containing gaboxadol and lithium, or a pharmaceutically acceptable salt of either or both compounds thereof, disintegrate rapidly to release the drug, which dissolves or disperses in the saliva. The drug may be absorbed in the oral cavity, e.g., sublingually, buccally, from the pharynx and esophagus or from other sections of gastrointestinal tract as the saliva travels down. In such cases, bioavailability can be significantly greater than that observed from conventional tablet dosage forms which travel to the stomach or intestines where drug can be released.

Intranasal forms enhance rapid uptake of gaboxadol and lithium, or a pharmaceutically acceptable salt of either or both compounds thereof, via the nasal and pulmonary system. Intranasal formulations of therapeutic agents are well known and those skilled in the art may adapt gaboxadol and lithium, or a pharmaceutically acceptable salt of either or both compounds thereof, to such a format. Design choices depend on whether the product will be a solution or suspension. Critical parameters include pH and buffer selection, osmolality, viscosity, excipient selection and choice of penetration enhancers or other components to enhance residence time in the nasal cavity. (See DPT Laboratories Ltd publications at www.dptlabs.com).

Where the compounds described herein or their pharmaceutically acceptable salts can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the synergistic combination of gaboxadol and lithium, or a pharmaceutically acceptable salt of either or both compounds thereof. In certain embodiments, the invention thus provides single unit dosage forms suitable for oral administration such as, but not limited to, tablets, capsules, gel caps, and caplets that are adapted for controlled- or sustained-release. In certain embodiments, the ingredients of a single unit dosage are supplied either separately or mixed together, for example, as a dry lyophilized-powder or water-free concentrate in a hermetically sealed container such as an ampule or sachet indicating the quantity of active agent. Where the compounds described herein or their pharmaceutically acceptable salts are to be administered by infusion, they can be dispensed, for example, with an infusion bottle containing sterile pharmaceutical grade water or saline. Where the compounds described herein or their pharmaceutically acceptable salts are administered by injection, an ampule of sterile water for injection or saline can be provided so that the ingredients can be mixed prior to administration.

8) Dose Regimen

The dosage regimen utilizing the synergistic combination of gaboxadol and lithium, or a pharmaceutically acceptable salt of either or both compounds thereof, can be selected in accordance with a variety of factors including type, species, age, weight, sex and medical condition of the subject; the severity of the condition to be treated; the route of administration; the renal or hepatic function of the subject; and the specific compound of the invention employed.

The synergistic combination of gaboxadol and lithium, or a pharmaceutically acceptable salt of either or both compounds thereof, can be administered in a single daily dose, or the total daily dosage can be administered in divided doses of two, three or four times daily.

In certain embodiments, combination of gaboxadol and lithium, or a pharmaceutically acceptable salt of either or both compounds thereof, can be administered in capsules having doses of, for example, 400 mg/20 mg, 400 mg/10 mg, 300 mg/10 mg, 300 mg/5 mg, 200 mg/5 mg or 100 mg/5 mg of lithium carbonate/gaboxadol.

In certain embodiments, the synergistic combination of gaboxadol and lithium is administered as a controlled release medicament. “Controlled release” as used herein is meant to encompass release of substance (e.g., lithium and/or gaboxadol) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals). “Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

In certain embodiments, an adult human diagnosed with bipolar disorder can be treated as follows:

### Acute Treatment:

| | Lithium | | |
|---|---|---|---|
| Mania or | Carbonate | Achieved lithium | Gaboxadol |
| hypomania | dose | serum level | monohydrate |
| per day | 50-1800 mg | 0.4 to 1.5 mmol/L | 5-150 mg |

### Chronic Prophylactic Treatment:

| | Lithium | | |
|---|---|---|---|
| | Carbonate | Achieved lithium | Gaboxadol |
| Maintenance | dose | serum level | monohydrate |
| per day | 50-900 mg | 0.2 to 1.2 mmol/L | 5-30 mg |

9) Kits

Kits for treating psychiatric disorders, e.g., depression, treatment resistant depression acute suicidality and bipolar disorder include a plurality of gaboxadol and lithium dosage forms and instructions for administering the dosage forms according to a predetermined dosage regimen. Here the predetermined dosing regimen may include administering doses of gaboxadol and lithium contemporaneously. The predetermined dosing regimen may provide that a dose of gaboxadol and lithium, or a pharmaceutically acceptable salt of either or both compounds thereof, be administered in the morning, e.g., at 6 am or between about 6 am and 9 am, and the lithium dose administered in the afternoon, e.g., at 12 pm (noon) or between about 12 pm and 3 pm, evening, e.g., at 6 pm or between about 6 pm and 9 pm, or late evening, e.g., at 12 am (midnight).

The kits may include a housing configured to organize the dosage forms according to the predetermined dosing regimen. For example, the housing may be configured to organize the plurality of dosage forms into morning dosage forms and evening dosage forms. In some variations, the housing may be configured to organize the plurality of gaboxadol and lithium dosage forms according to a rapidly or a gradually decreasing dosing regimen. In yet further variations, the housing may be configured to organize the gaboxadol and lithium dosage forms according to the day of the week to be taken.

The kits may include a housing configured to organize the dosage forms according to the predetermined dosing regimen. For example, the housing may be configured to organize the plurality of dosage forms into morning dosage forms and evening dosage forms. In some variations, the housing may be configured to organize the plurality of dosage forms according to a rapidly or a gradually decreasing dosing regimen. In yet further variations, the housing may be configured to organize the dosage forms according to the day of the week to be taken. The kits may also be tailored to treat particular bipolar conditions or subtypes. For example, the kits may be tailored to treat bipolar I disorder, bipolar II disorder, mixed bipolar disorders, rapidly-cycling bipolar disorder, acute mania, drug-induced mania, hypomania, cyclothymia, or combinations thereof.

Although this invention has been described with a certain degree of particularity, it is to be understood that the present disclosure has been made only by way of illustration and that numerous changes in the details of construction and arrangement of parts may be resorted to without departing from the spirit and the scope of the invention.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict between the present explicit disclosure and a document incorporated by reference, the present explicit disclosure shall be the operative disclosure.

### EXAMPLES

### Example 1: Whole-Brain Drug Screening Platform

Many preclinical assays are currently used to try to elucidate or predict the clinical effects of new drugs on the brain. These include in vitro high-content screening (HCS) assays that measure a drug's pharmacokinetics for specific molecular target(s) and its effect(s) in simple cellular assays, in vivo assays that measure global responses at relatively low resolution (PET/CT, PET/MRI, fMRI) or local responses at high, cellular resolution (electrophysiology or two-photon imaging), and behavioral assays that measure animal's performance in various tasks (Jain and Heutink, 2010; Judenhofer et al., 2008; Markou et al., 2009). Despite a great deal of effort put into preclinical research, the clinical effects of drugs continue to be unpredictable, plaguing the drug development pipeline and resulting in a >90% failure rate in clinical trials (Pammolli et al., 2011).

To assess neuronal activity in regions less accessible to live imaging, Immediate Early Genes (IEGs) such as c-fos, whose expression levels reflect recent changes in neuronal activity, have been used as proxies. The first in vivo example of induced c-fos expression in neurons was reported in the dorsal horn of the spinal cord following a nociceptive stimulus (Hunt et al., 1987). Since then, the up-regulation of expression of IEGs like c-fos, arc, egr-1, fosb and npas4 have been used as a surrogates for neuronal activity in most neuronal systems and in most regions of the brain.

The disclosed unique and novel approach to preclinical testing of psychiatric drugs is based on the proposition that a direct readout of drug-evoked brain activation or inhibition in an animal is the most relevant preclinical assay, because psychiatric drugs exert their effects via activation or inhibition of specific neural circuits and cell types in the brain.

Importantly, in contrast to the limitations of existing in vivo methods to measure brain activation, such as PET/CT, PET/MRI and phMRI that suffer from low spatial resolution, or electrophysiology or two-photon imaging that suffer from a limited spatial scope, the disclosed “pharmacomapping” approach enables the direct visualization and measurement of drug-evoked brain activation or inhibition across the entire mouse brain at an unprecedented single cell resolution. This method called “pharmacomapping” (implemented as a fee for service by CRO Certerra, Inc. Farmingdale, N.Y.) is based on a proprietary and largely automated drug-screening platform that comprises whole-brain detection of drug-evoked neuronal activation represented by drug-evoked expression of the immediate early gene (IEG) c-fos. Until now, the detection of c-fos as a marker of brain activation required time-consuming, laborious methods such as in situ hybridization or immunohistochemistry in brain tissue sections, followed by mounting the sections on microscopic slides, manual imaging, and largely visual quantification. Nevertheless, over the last two decades a number of studies used these methods to test drug-evoked activity in the mouse or rat brain for various psychoactive medications, including antipsychotics, antidepressants, stimulants and anxiolytics (Engber et al., 1998; Salminen et al., 1996; SEMBA et al., 1996; Slattery et al., 2005; Sumner et al., 2004). These studies, even though typically examining only a few brain regions at a time, represent a validation for the concept of using c-fos expression in the rodent brain in psychoactive drug screening (Sumner et al., 2004).

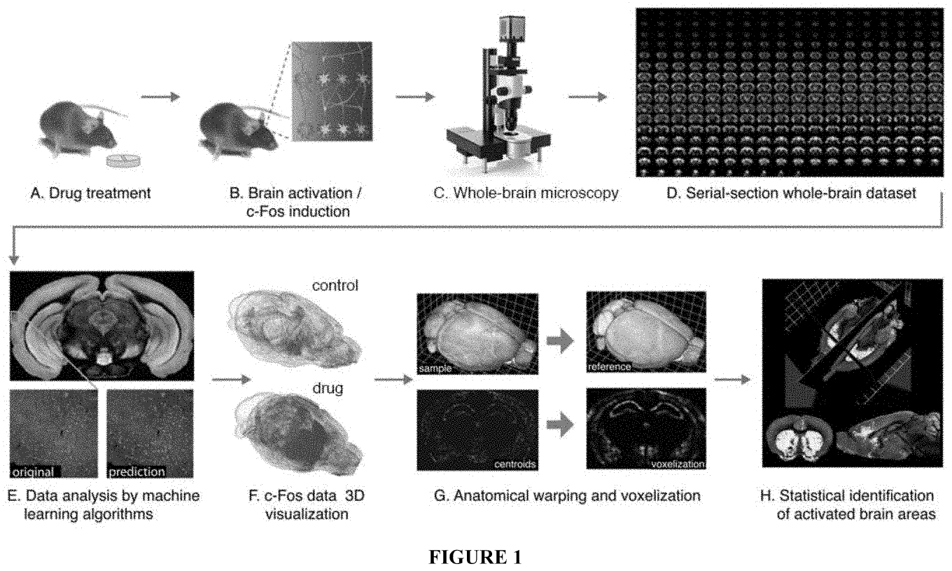

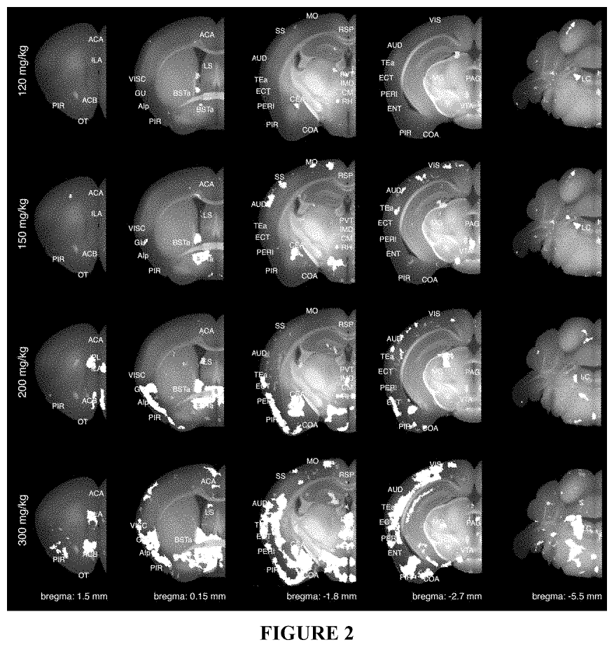

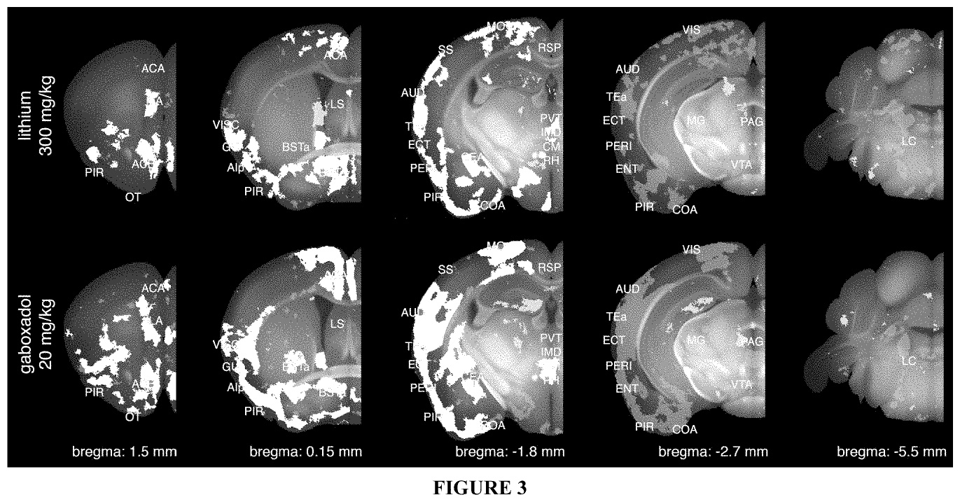

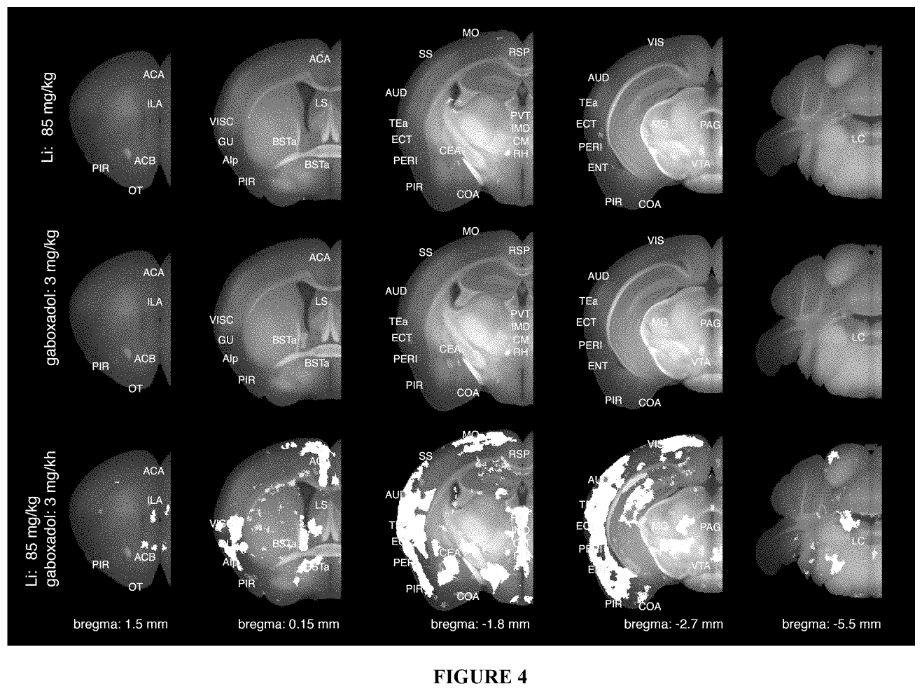

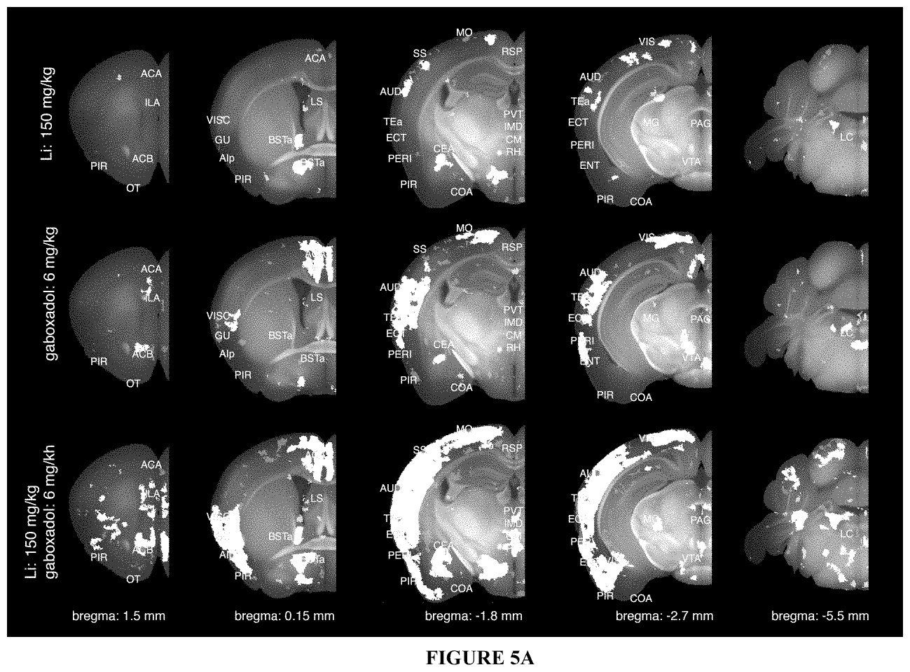

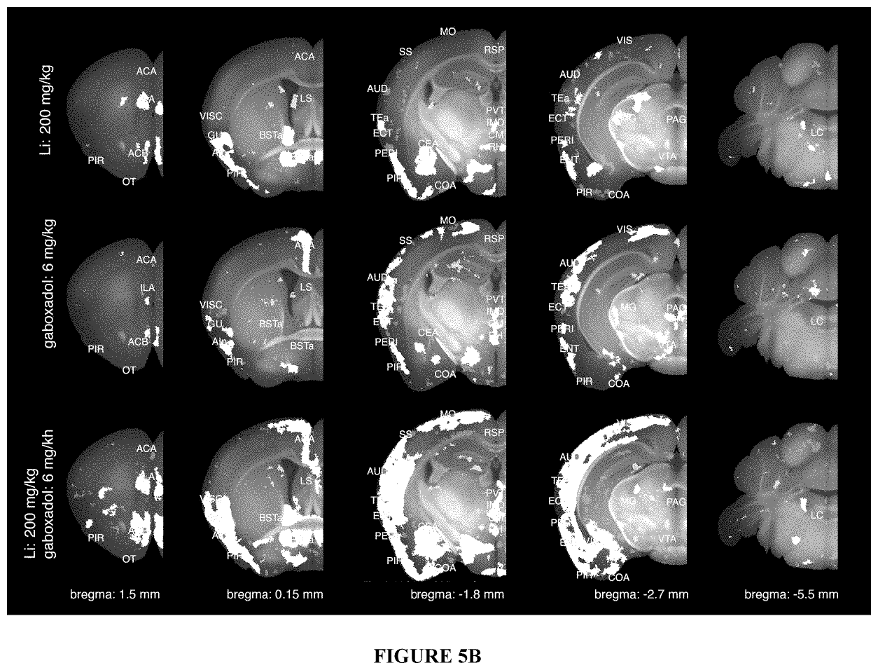

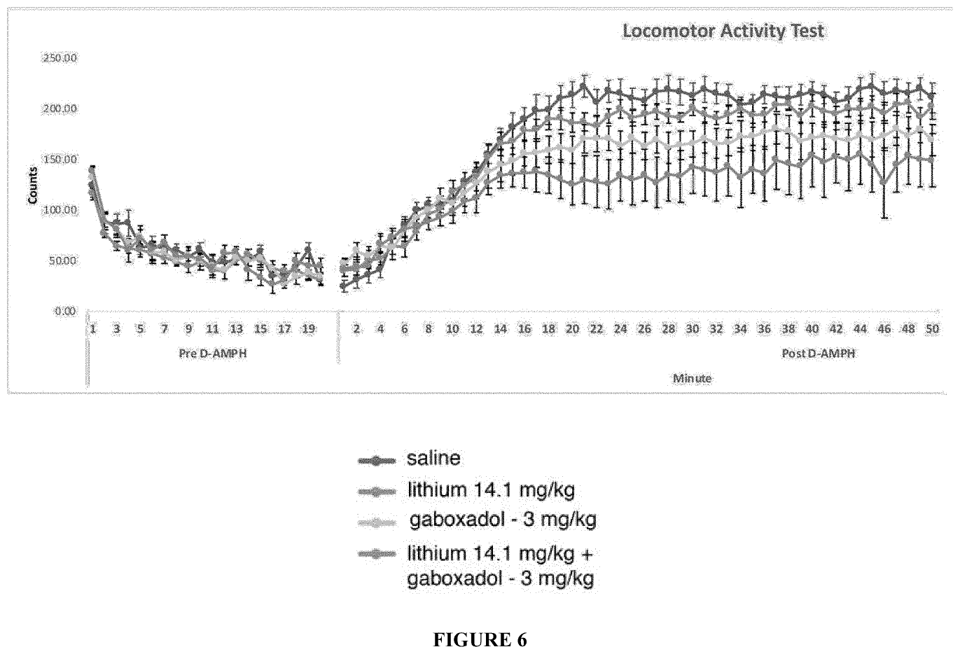

In contrast to the older methods, the pharmacomapping method uses largely automated and standardized whole-brain immunostaining and brain clearing together with advanced microscopy (light-sheet fluorescence microscopy, LSFM), computational (e.g. machine learning) and statistical methods (FIG. 1; see, for example, the published U.S. Patent Application No. 2014/0297199, the content of which is incorporated by reference herein in its entirety). The first generation of this platform used serial two-photon tomography (STPT) as a method for imaging and c-fos-GFP mice expressing green fluorescent protein (GFP) under the control of the c-fos promoter (see, e.g., published U.S. Patent Application No. 2014/0297199, the content of which is incorporated by reference herein in its entirety).

The second generation of the pharmacomapping platform currently employed by Certerra uses whole-brain immunostaining and clearing procedure named iDISCO+ and whole-brain imaging by light-sheet fluorescence microscopy to detect c-fos-positive neurons in wild type mice (Renier et al., 2016). The pharmacomapping platform thus uses the well-established concept of c-fos expression as a cellular marker of neuronal activation and applies it as a standardized and highly quantitative whole-brain assay capable of generating detailed and reproducible drug-evoked whole-brain activation patterns, called Pharmacomaps®.

### Example 2: Mapping the Brain Activation Underlying the Therapeutical Action of Lithium in Psychiatry

To understand the mechanism of action of lithium across the entire brain, the aforementioned pharmacomapping technique was used to map lithium evoked brain activation in response to the following doses in the mouse (mg/kg): 120, 150, 200, and 300 which corresponds approximately to human equivalent doses (mg): 600, 750, 1000, and 1500. These experiments revealed a dose-dependent increase in the pattern of brain activation that included a modest to moderate activation of a few structures at 120 and 150 mg/kg and a considerably broader activation at 200 and 400 mg/kg (FIG. 2). The activation patterns observed with a lithium dose of 120 and 150 mg/kg included the anterior portion of the bed nuclei stria terminalis (BSTa), central amygdala (CEA), and locus coeruleus (LC) (FIG. 2, top rows). The same structures were also prominently activated by lithium at 200 and 300 mg/kg, in addition to an activation of the prelimbic (PL) and infralimbic (ILA) cortex, piriform cortex (PIR) and nucleus accumbens (ACB) at bregma 1.5 mm, the gustatory (GU), agranular insular (AIp) cortical areas, motor (MO), somatosensory (SS), auditory (AUD), temporal associational (TEa), perirhinal (PERI) and entorhinal cortex, as well as midline thalamic nuclei, including the paraventricular nucleus (PVT), intermediodorsal nucleus (IMB), central medial nucleus (CM), and rhomboid nucleus (RH) at bregma 0.15 to −1.8 mm, and the visual (VIS), ectorhinal (ECT) TEa, AUD, PERI and ENT cortical areas, as well as medial geniculate complex (MG) cortical amygdala at bregma 2.7 mm (FIG. 2, bottom rows).

### Example 3: The Pattern of Lithium-Induced C-Fos Activation Closely Matches that of the Gaba-A Agonist, Gaboxadol

Mapping the effect of lithium across the mouse brain using the aforementioned pharmacomapping platform permits a direct comparison of the lithium-evoked brain activation pattern with that of other test compounds. Strikingly, the pharmacomap pattern evoked by high dose 300 mg/kg lithium closely matched that of gaboxadol at 20 mg/kg, including c-fos activation of 1) a broad cortical activation comprising motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, claustrum (CLA), as well as 2) subcortical activation comprising hippocampal CA1 region, the bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of the thalamus, paraventricular hypothalamic nucleus (PVH), dorsomedial nucleus of the hypothalamus (DMH), tuberomammillary nucleus (TM), parasubthalamic nucleus (PSTN) and subthalamic nucleus (STN), parabrachial nucleus, locus coeruleus (LC), and nucleus of the solitary tract (NTS) (FIG. 3).

This discovery is all the more surprising because gaboxadol and lithium are structurally unrelated molecules: gaboxadol is an agonist at the delta-subunit containing GABAergic receptors that are believed to constitute an extra-synaptic population of inhibitory GABA-A receptors in the brain, while the mechanism of action for lithium in the brain remains not well established, though several studies have suggested involvement in signaling cascades downstream of inhibition of glycogen synthase kinase 3-beta, leading to neurotrophic effects and enhanced neuroplasticity and cellular resilience (Won and Kim, 2017). Thus, the discovery that lithium evokes brain-wide activation that matches the pattern seen with gaboxadol is entirely unexpected and could not have been predicted based on reports in the scientific literature.

### Example 4: Synergy Between Lithium and Gaboxadol in a Pharmacomapping Assay

The similarity of the lithium and gaboxadol pharmacomaps suggested that the initial compound-specific signaling events lead to a common downstream brain circuit activation. To test whether gaboxadol and lithium are able to act in synergy with one another, two low doses of each compound were combined under conditions where the dose of each compound on their own did not evoke any acute brain activation. As shown in FIG. 4, neither gaboxadol at 3 mg/kg nor lithium at 85 mg/kg alone evoked any brain activation detectable using the pharmacomapping assay (FIG. 4, top two rows). However, the combination of gaboxadol at 3 mg/kg+lithium at 85 mg/kg elicited a strong activation of a number of areas that were also activated by each drug individually when administered at the dose of 20 and 300 mg/kg, respectively, as described above (FIG. 4, top bottom row).

Furthermore, this synergy is not limited to the low doses of lithium (<100 mg/kg) and gaboxadol (<5 mg/kg), but is also seen in combination of two compounds at higher doses, such as gaboxadol at 6 mg/kg and lithium at 150 mg/kg (FIG. 5A) or gaboxadol at 6 mg/kg and lithium at 200 mg/kg (FIG. 5B). At the higher drug combinations, in addition to synergy, additive effects between gaboxadol and lithium are also observed.

Taken together, these data clearly demonstrate that lithium and gaboxadol can synergize in their brain activation action, establishing that a combination therapy is an effective strategy to achieve lithium efficacy while lowering lithium induced side effects. It is also important to note that gaboxadol has been tested in clinical trials and found to have no adverse side-effects at human doses equivalent to the mouse 6 mg/kg dose used in the current study. For example, in the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia. The development was discontinued after the compound failed to show significant effects in sleep onset and sleep maintenance in a three-month efficacy study.

### Example 5: Lithium and Gaboxadol Synergize in an Amphetamine Induced Rodent Model of Mania

The stimulant d-amphetamine-induced hyperactivity has been used as a therapeutically predictive rodent test of mania, as pretreatment with lithium was shown to suppress the amphetamine-induced hyperlocomotion (Berggren et al., 1978; Cappeliez and Moore, 1990; Kato et al., 2007). The brain activation synergy action between lithium and gaboxadol seen in the pharmacomapping experiments described above suggests that the two molecules should also synergize in the d-amphetamine test, leading to an enhanced suppression of hyperlocomotion than seen with either molecule alone.

As shown in FIG. 6, while pretreatment with a sub-effective dose (14.1 mg/kg) of lithium had no behavioral effect, pretreatment with the 14.1 mg/kg sub-effective dose of lithium in combination with a low dose of 3 mg/kg gaboxadol had a synergistic effect in suppressing amphetamine-induced hyperlocomotion in comparison to either lithium or gaboxadol alone.

### CITED REFERENCES

Abou-Saleh, M. T., Muller-Oerlinghausen, B., and Coppen, A. J. (2017). Lithium in the episode and suicide prophylaxis and in augmenting strategies in patients with unipolar depression. International journal of bipolar disorders 5, 11.
Ando, S., Koike, S., Shimodera, S., Fujito, R., Sawada, K., Terao, T., Furukawa, T. A., Sasaki, T., Inoue, S., and Asukai, N. (2017). Lithium Levels in Tap Water and the Mental Health Problems of Adolescents: An Individual-Level Cross-Sectional Survey. The Journal of clinical psychiatry 78, e252-e256.
Andrade Nunes, M., Araujo Viel, T., and Sousa Buck, H. (2013). Microdose lithium treatment stabilized cognitive impairment in patients with Alzheimer's disease. Current Alzheimer Research 10, 104-107.
Association, A. P. (2002). Practice guideline for the treatment of patients with bipolar disorder (revision) (American Psychiatric Pub).
Baldessarini, R. J., Tondo, L., Davis, P., Pompili, M., Goodwin, F. K., and Hennen, J. (2006). Decreased risk of suicides and attempts during long-term lithium treatment: a meta-analytic review. Bipolar disorders 8, 625-639.
Baldessarini, R. J., Tondo, L., and Hennen, J. (2003). Lithium treatment and suicide risk in major affective disorders: update and new findings. Journal of clinical psychiatry 64, 44-52.
Berggren, U., Tallstedt, L., Ahlenius, S., and Engel, J. (1978). The effect of lithium on amphetamine-induced locomotor stimulation. Psychopharmacology 59, 41-45.
Bersani, G., Quartini, A., Zullo, D., and Iannitelli, A. (2016). Potential neuroprotective effect of lithium in bipolar patients evaluated by neuropsychological assessment: preliminary results. Human Psychopharmacology: Clinical and Experimental 31, 19-28.
Cappeliez, P., and Moore, E. (1990). Effects of lithium on an amphetamine animal model of bipolar disorder. Progress in Neuro-Psychopharmacology and Biological Psychiatry 14, 347-358.
Carter, L., Zolezzi, M., and Lewczyk, A. (2013). An updated review of the optimal lithium dosage regimen for renal protection. The Canadian Journal of Psychiatry 58, 595-600.
Cipriani, A., Hawton, K., Stockton, S., and Geddes, J. R. (2013). Lithium in the prevention of suicide in mood disorders: updated systematic review and meta-analysis. Bmj 346, f3646.
Cipriani, A., Pretty, H., Hawton, K., and Geddes, J. R. (2005). Lithium in the prevention of suicidal behavior and all-cause mortality in patients with mood disorders: a systematic review of randomized trials. American Journal of Psychiatry 162, 1805-1819.
Davis, J., Desmond, M., and Berk, M. (2018a). Lithium and nephrotoxicity: a literature review of approaches to clinical management and risk stratification. BMC nephrology 19, 305.
Davis, J., Desmond, M., and Berk, M. (2018b). Lithium and nephrotoxicity: Unravelling the complex pathophysiological threads of the lightest metal. Nephrology 23, 897-903.
Deligiannidis, K. M., Byatt, N., and Freeman, M. P. (2014). Pharmacotherapy for mood disorders in pregnancy: a review of pharmacokinetic changes and clinical recommendations for therapeutic drug monitoring. Journal of clinical psychopharmacology 34, 244.
Engber, T. M., Koury, E. J., Dennis, S. A., Miller, M. S., Contreras, P. C., and Bhat, R. V. (1998). Differential patterns of regional c-Fos induction in the rat brain by amphetamine and the novel wakefulness-promoting agent modafinil. Neuroscience letters 241, 95-98.
Gelenberg, A. J., Kane, J. M., Keller, M. B., Lavori, P., Rosenbaum, J. F., Cole, K., and Lavelle, J. (1989). Comparison of standard and low serum levels of lithium for maintenance treatment of bipolar disorder. New England Journal of Medicine 321, 1489-1493.
Grandjean, E. M., and Aubry, J.-M. (2009). Lithium: updated human knowledge using an evidence-based approach. CNS drugs 23, 397-418.
Horton, S., Tuerk, A., Cook, D., Cook, J., and Dhurjati, P. (2012). Maximum recommended dosage of lithium for pregnant women based on a PBPK model for lithium absorption. Advances in bioinformatics 2012.
Hunt S P, Pini A and Evan G (1987) Induction of c-fos-like protein in spinal cord neurons following sensory stimulation. Nature 13-19; 328(6131):632-4.
Ivkovic, A., and Stern, T. A. (2014). Lithium-induced neurotoxicity: clinical presentations, pathophysiology, and treatment. Psychosomatics 55, 296.
Jain, S., and Heutink, P. (2010). From single genes to gene networks: high-throughput-high-content screening for neurological disease. Neuron 68, 207-217.
Judenhofer, M. S., Wehrl, H. F., Newport, D. F., Catana, C., Siegel, S. B., Becker, M., Thielscher, A., Kneilling, M., Lichy, M. P., and Eichner, M. (2008). Simultaneous PET-MM: a new approach for functional and morphological imaging. Nature medicine 14, 459-465.
Kato, T., Kubota, M., and Kasahara, T. (2007). Animal models of bipolar disorder. Neuroscience & Biobehavioral Reviews 31, 832-842.
Kessing, L. V., Bauer, M., Nolen, W. A., Severus, E., Goodwin, G. M., and Geddes, J. (2018). Effectiveness of maintenance therapy of lithium vs other mood stabilizers in monotherapy and in combinations: a systematic review of evidence from observational studies. Bipolar disorders 20, 419-431.
Ljubicic, D., Letica-Crepulja, M., Vitezic, D., Bistrovic, I. L., and Ljubicic, R. (2008). Lithium treatments: single and multiple daily dosing. The Canadian Journal of Psychiatry 53, 323-331.
Madhusudhan, B. (2014). Nonconvulsive status epilepticus and Creutzfeldt-Jakob-like EEG changes in a case of lithium toxicity. Epilepsy & behavior case reports 2, 203-205.
Markou, A., Chiamulera, C., Geyer, M. A., Tricklebank, M., and Steckler, T. (2009). Removing obstacles in neuroscience drug discovery: the future path for animal models. Neuropsychopharmacology 34, 74-89.
Megarbane, B., Hanak, A.-S., and Chevillard, L. (2014). Lithium-related neurotoxicity despite serum concentrations in the therapeutic range: risk factors and diagnosis. Shanghai archives of psychiatry 26, 243.
Öhlund, L., Ott, M., Oja, S., Bergqvist, M., Lundqvist, R., Sandlund, M., Renberg, E. S., and Werneke, U. (2018). Reasons for lithium discontinuation in men and women with bipolar disorder: a retrospective cohort study. BMC psychiatry 18, 37.
Pammolli, F., Magazzini, L., and Riccaboni, M. (2011). The productivity crisis in pharmaceutical R&D. Nat Rev Drug Discov 10, 428-438.
Post, R. M. (2018). The new news about lithium: An underutilized treatment in the United States. Neuropsychopharmacology 43, 1174.
Quartini, A., Iannitelli, A., and Bersani, G. (2016). Lithium: from mood stabilizer to putative cognitive enhancer. Neural regeneration research 11, 1234.
Rej, S., Beaulieu, S., Segal, M., Low, N. C., Mucsi, I., Holcroft, C., Shulman, K., and Looper, K. J. (2014). Lithium dosing and serum concentrations across the age spectrum: from early adulthood to the tenth decade of life. Drugs & aging 31, 911-916.
Renier, N., Adams, E. L., Kirst, C., Wu, Z., Azevedo, R., Kohl, J., Autry, A. E., Kadiri, L., Umadevi Venkataraju, K., Zhou, Y., et al. (2016). Mapping of Brain Activity by Automated Volume Analysis of Immediate Early Genes. Cell 165, 1789-1802.
Roberts, E., Cipriani, A., Geddes, J. R., Nierenberg, A. A., and Young, A. H. (2017). The evidence for lithium in suicide prevention. The British Journal of Psychiatry 211, 396-396.
Rybakowski, J. K., and Suwalska, A. (2010). Excellent lithium responders have normal cognitive functions and plasma BDNF levels. International Journal of Neuropsychopharmacology 13, 617-622.
Salminen, O., Lahtinen, S., and Ahtee, L. (1996). Expression of Fos protein in various rat brain areas following acute nicotine and diazepam. Pharmacology Biochemistry and Behavior 54, 241-248.
Sani, G., Perugi, G., and Tondo, L. (2017). Treatment of bipolar disorder in a lifetime perspective: is lithium still the best choice? Clinical drug investigation 37, 713-727.
Schou, M., AMDISEN, A., and TRAP-JENSEN, J. (1968). Lithium poisoning. American Journal of Psychiatry 125, 520-527.
Scott, J., Geoffroy, P. A., Sportiche, S., Brichant-Petit-Jean, C., Gard, S., Kahn, J.-P., Azorin, J.-M., Henry, C., Etain, B., and Bellivier, F. (2017). Cross-validation of clinical characteristics and treatment patterns associated with phenotypes for lithium response defined by the Alda scale. Journal of affective disorders 208, 62-67.
SEMBA, J. I., SAKAI, M., MIYOSHI, R., MATAGA, N., FUKAMAUCHI, F., and KITO, S. (1996). Differential expression of c-fos mRNA in rat prefrontal cortex, striatum, N. accumbens and lateral septum after typical and atypical antipsychotics: an in situ hybridization study. Neurochemistry international 29, 435-442.
Severus, E., Taylor, M. J., Sauer, C., Pfennig, A., Ritter, P., Bauer, M., and Geddes, J. R. (2014). Lithium for prevention of mood episodes in bipolar disorders: systematic review and meta-analysis. International journal of bipolar disorders 2, 15.
Singh, L. K., Nizamie, S. H., Akhtar, S., and Praharaj, S. K. (2011). Improving tolerability of lithium with a once-daily dosing schedule. American journal of therapeutics 18, 288-291.
Slattery, D. A., Morrow, J. A., Hudson, A. L., Hill, D. R., Nutt, D. J., and Henry, B. (2005). Comparison of alterations in c-fos and Egr-1 (zif268) expression throughout the rat brain following acute administration of different classes of antidepressant compounds. Neuropsychopharmacology 30, 1278.
Sumner, B. E., Cruise, L. A., Slattery, D. A., Hill, D. R., Shahid, M., and Henry, B. (2004). Testing the validity of c-fos expression profiling to aid the therapeutic classification of psychoactive drugs. Psychopharmacology (Berl) 171, 306-321.
Tiihonen, J., Lahteenvuo, M., Hoti, F., Vattulainen, P., Taipale, H., and Tanskanen, A. (2016). Real-World Effectiveness of Pharmacological Treatments in Severe Unipolar Depression in a Nationwide Cohort of 123,712 Patients. American College of Neuropsychopharmacology: Hollywood, Fla.
Toffol, E., Hatonen, T., Tanskanen, A., Lönnqvist, J., Wahlbeck, K., Joffe, G., Tiihonen, J., Haukka, J., and Partonen, T. (2015). Lithium is associated with decrease in all-cause and suicide mortality in high-risk bipolar patients: a nationwide registry-based prospective cohort study. Journal of affective disorders 183, 159-165.
Vita, A., De Peri, L., and Sacchetti, E. (2015). Lithium in drinking water and suicide prevention: a review of the evidence. International clinical psychopharmacology 30, 1-5.
Wesseloo, R., Wierdsma, A. I., van Kamp, I. L., Munk-Olsen, T., Hoogendijk, W. J., Kushner, S. A., and Bergink, V. (2017). Lithium dosing strategies during pregnancy and the postpartum period. The British Journal of Psychiatry 211, 31-36.
Won, E., and Kim, Y.-K. (2017). An oldie but goodie: lithium in the treatment of bipolar disorder through neuroprotective and neurotrophic mechanisms. International journal of molecular sciences 18, 2679.

What is claimed is:

1. A pharmaceutical composition comprising a synergistic combination of compounds present in synergistically effective amounts, wherein the synergistic combination is a combination consisting of gaboxadol and lithium, or a pharmaceutically acceptable salt of either or both compounds thereof.

2. The pharmaceutical composition of claim 1, wherein the lithium is a sub-standard daily dose of lithium.

3. The pharmaceutical composition of claim 2, wherein the sub-standard dose of lithium, when administered daily to a subject in need thereof, is below the medically recommended dose for treating bipolar disorder, depression, treatment-resistant depression, or suicidality.

4. The pharmaceutical composition of claim 2, wherein an animal equivalent of the sub-standard dose of lithium is ineffective at activating c-fos signalling in an animal model's brain as measured by Pharmacomapping.

5. The pharmaceutical composition of claim 2, wherein a human equivalent of the sub-standard dose of lithium is in a range from about 50 to about 600 mg lithium carbonate/day.

6. The pharmaceutical composition of claim 1, wherein the gaboxadol is a low to medium dose of gaboxadol.

7. The pharmaceutical composition of claim 6, wherein an animal equivalent of the low dose of gaboxadol is ineffective at activating c-fos signalling in an animal model's brain as measured by Pharmacomapping.

8. The pharmaceutical composition of claim 6, wherein a human equivalent of the low dose of gaboxadol is in a range from about 5 to about 15 mg gaboxadol/day.

9. The pharmaceutical composition of claim 6, wherein the medium dose of gaboxadol is in a range from about 15 to about 30 mg gaboxadol/day.

10. The pharmaceutical composition of claim 1, wherein the lithium is a standard dose of lithium.

11. The pharmaceutical composition of claim 10, wherein the standard dose of lithium is in a range from about 600 to about 1800 mg, with a maximum daily dose of 2400 mg, of lithium carbonate/day.

12. The pharmaceutical composition of claim 1, wherein the gaboxadol is a high dose of gaboxadol.

13. The pharmaceutical composition of claim 12, wherein an animal equivalent of the high dose of gaboxadol is effective at activating broad c-fos signalling in an animal model's brain.

14. The pharmaceutical composition of claim 12, wherein a human equivalent of the high dose of gaboxadol is in a range from about 30 to about 300 mg gaboxadol/day.

15. The pharmaceutical composition of claim 1, wherein animal equivalent doses of lithium and gaboxadol administered daily to an animal model in need thereof are synergistically effective at activating c-fos signalling in at least one region of an animal model's brain selected from the group consisting of 1) a broad cortical activation comprising motor (MO), gustatory (GU), visceral (VISC), agranular insular (AI), somatosensory (SS), auditory, visual (VIS), auditory (AUD), prelimbic (PL) and infralimbic (ILA), retrosplenial (RSP), parietal (PTL), temporal associational (TEa), ectorhinal (ECT), entorhinal (ENT), perirhinal (PERI), piriform (PIR), and anterior cingulate (ACA) cortex, claustrum (CLA), as well as 2) subcortical activation comprising hippocampal CA1 region, bed nuclei stria terminalis (BST), central amygdala (CEA), cortical amygdala (COA), basolateral and basomedial amygdala (BLA and BMA), medial amygdala (MEA), thalamic ventral posteromedial nucleus (VPM), subparafascicular nucleus (SPF), medial geniculate complex (MG), suprageniculate nucleus (SGN), nucleus of reunions (RE), rhomboid nucleus (RH), and central medial nucleus (CM) of a thalamus, paraventricular hypothalamic nucleus (PVH), dorsomedial nucleus of a hypothalamus (DMH), tuberomammillary nucleus (TM), parasubthalamic nucleus (PSTN) and subthalamic nucleus (STN), parabrachial nucleus, locus coeruleus (LC), and nucleus of a solitary tract (NTS).

16. The pharmaceutical composition of claim 1, wherein the gaboxadol and lithium, when administered daily to a subject in need thereof, act synergistically to treat the subject's psychiatric disorder selected from the group consisting of bipolar disorder, depression, treatment resistant depression, and acute suicidality.

17. The pharmaceutical composition of claim 16, wherein the treatment of the subject's psychiatric disorder is effective at improving a score of at least one psychiatric rating scale specific for bipolar disorder, depression, treatment resistant depression, or suicidality.

18. The pharmaceutical composition of claim 1, wherein the gaboxadol and lithium, when administered to a subject diagnosed with bipolar depression, unipolar depression or treatment resistant depression are synergistically effective at increasing the subject's Montgomery-Asberg Depression Rating Scale (MADRS) score.

19. The pharmaceutical composition of claim 1, wherein the gaboxadol and lithium, when administered to a subject in need thereof, are synergistically effective at increasing a score of at least one psychiatric rating scale specific for bipolar disorder, depression, treatment resistant depression, or suicidality.

20. The pharmaceutical composition of claim 1, wherein the lithium, when administered daily to a subject in need thereof, is in an amount sufficient to maintain the subject's serum level of lithium in a range of about 0.4 to about 1.2 mmol/L.

21. The pharmaceutical composition of claim 2, wherein the lithium, when administered daily to a subject in need thereof, is in an amount sufficient to maintain the subject's serum level of lithium in a range of about 0.2 to about 0.8 mmol/L.

22. The pharmaceutical composition of claim 1, wherein the pharmaceutical composition is in a form of a single tablet for oral consumption.

23. The pharmaceutical composition of claim 1, wherein the pharmaceutical composition is in a form of a controlled release formulation.

24. The pharmaceutical composition of claim 1, further comprising one or more inert pharmaceutically acceptable excipients.

25. The pharmaceutical composition of claim 1, wherein the pharmaceutical composition is in a form of a single dosage unit having separate compartments for the lithium and gaboxadol or a pharmaceutically acceptable salt of either or both compounds thereof.

26. A kit comprising the pharmaceutical composition of claim 1.

27. A method for treating a subject in need thereof comprising administering the pharmaceutical composition of claim 1.

28. The method of claim 27, wherein the subject is diagnosed with a psychiatric disorder.

29. The method of claim 28, wherein the psychiatric disorder is chosen from bipolar disorder, depression, treatment-resistant depression, or acute suicidality.

30. The method of claim 28, wherein the pharmaceutical composition reduces adverse side effects selected from the group consisting of nephrotoxicity, nephrogenic diabetes insipidus, chronic kidney disease, diarrhea, hand tremor, increased thirst, increased urination, vomiting, weight gain, impaired memory, poor concentration, drowsiness, muscle weakness, hair loss, acne and decreased thyroid function.

31. A method for treating a human diagnosed with bipolar disorder, depression, or acute suicidality comprising administering a synergistic combination of gaboxadol at a dose ranging from about 5 to about 300 mg/day, contemporaneously with lithium
a) at a dose from about 50 mg to about 1800 mg lithium carbonate; or
b) from about 0.8 mg/kg to about 30 mg/kg lithium carbonate; or
c) in an amount sufficient to achieve a lithium serum concentration of about 0.2 to 1.2 mmol/L;
wherein the combination dose of gaboxadol and lithium is administered at least once per day.

32. A method for treating a human diagnosed with an acute form of bipolar disorder, depression, or suicidality comprising administering a synergistic combination of gaboxadol at a dose in a range of from about 5 mg to about 50 mg/day, contemporaneously with lithium
a) at a dose of from about 50 mg to about 900 mg lithium carbonate/day; or
b) in an amount sufficient to achieve a lithium serum concentration of 0.2 to 1.0 mmol/L;
wherein the combination dose of gaboxadol and lithium is administered at least once per day.

33. A method for treating a patient diagnosed with a chronic form of bipolar disorder, depression, or suicidality comprising administering a synergistic combination of gaboxadol at a dose in a range of from about 5 mg to about 30 mg/day, contemporaneously with lithium
a) at a dose of from about 50 mg to about 600 mg lithium carbonate; or
a) in an amount sufficient to achieve a lithium serum concentration of about 0.2 to 0.8 mmol/L;
wherein the combination dose of gaboxadol and lithium is administered at least once per day.

34. The use of a synergistic combination of gaboxadol and lithium, or a pharmaceutically acceptable salt of either or both compounds thereof, for reducing reducing one or symptoms of bipolar disorder, depression, or suicidality.

35. The use of a synergistic combination of gaboxadol and lithium, or a pharmaceutically acceptable salt of either or both compounds thereof, in the manufacture of a medicament for reducing one or symptoms of bipolar disorder, depression, or suicidality.
